(19) United States
(12) Patent Application Publication
(10) Pub. No.: US 2020/0345533 A1
(43) Pub. Date: Nov. 5, 2020

(54) WEARABLE PATCH FOR REDUCING SNORING ACTIVITY

(71) Applicant: LIFE PATCH INTERNATIONAL, Irvine, CA (US)

(72) Inventors: Romilda Olszewski BOLLELLA, Irvine, CA (US); Donald BOLLELLA, Irvine, CA (US)

(21) Appl. No.: 16/864,863

(22) Filed: May 1, 2020

Related U.S. Application Data
(60) Provisional application No. 62/842,472, filed on May 2, 2019.

(51) Int. Cl. A61F 5/56

(52) U.S. Cl. CPC A61F 5/56; A61H 1/005

(57) ABSTRACT

A patch can include a wearable component configured to allow the patch to be worn on a user, and a sensor component implemented relative to the wearable component. The sensor component can include a transducer configured to generate an electrical signal based on a snoring sound. In some embodiments, such a patch can be utilized in a system for reducing snoring of the user. The system can include a patch generating a first signal indicative of a snoring sound, and a control unit configured to generate a second signal based on the first signal, with the second signal being configured to allow actuation of a stimulus that reduces snoring activity associated with the snoring sound. The system can further include a sleep adjustment device in communication with the control unit and configured to generate the stimulus based on the second signal.

### CROSS-REFERENCE TO RELATED APPLICATION(S)

This application claims priority to U.S. Provisional Application No. 62/842,472 filed May 2, 2019, entitled WEARABLE PATCH FOR REDUCING SNORING ACTIVITY, the disclosure of which is hereby expressly incorporated by reference herein in its respective entirety.

### BACKGROUND

### Field

The present disclosure relates to a wearable patch for reducing snoring activity of a user.

### Description of the Related Art

Aside from an indication of a person's health condition, snoring by the person can affect others. For example, another person sleeping or attempting to sleep next to the snoring person can be woken up or have difficulty falling asleep due to the snoring noise.

### SUMMARY

In accordance with some implementations, the present disclosure relates to a system for reducing snoring activity. The system includes a patch configured to be secured to a skin of a user and generate a first signal indicative of a snoring sound. The system further includes a control unit configured to generate a second signal based on the first signal. The second signal is configured to allow actuation of a stimulus that reduces snoring activity associated with the snoring sound.

In some embodiments, the user having the patch can be a sleeping person generating the snoring sound.

In some embodiments, the control unit can be implemented as part of the wearable patch.

In some embodiments, the control unit can include a device separated from the patch. Such a device can be a wireless device, and the first signal can include a first wireless signal transmitted from the patch to the wireless device. The wireless device can be, for example, a smartphone.

In some embodiments, the system can further include a sleep adjustment device in communication with the control unit and configured to generate the stimulus based on the second signal.

In some embodiments, the sleep adjustment device can be part of an adjustable bed. The stimulus generated by the sleep adjustment device can result in, for example, a change in an angle of a mattress of the adjustable bed.

In some embodiments, the sleep adjustment device can be part of a mattress. In some embodiments, such a mattress can be configured to be supported by a fixed frame. In some embodiments, the sleep adjustment device can be implemented as an integral part of the mattress, or as a pad configured to be placed on the mattress.

In some embodiments, the stimulus generated by the sleep adjustment device can be a mechanical stimulus. In some embodiments, the sleep adjustment device can include a plurality of actuator elements implemented such that one or more selected ones of the actuator elements are activated based on the second signal. Each of the actuator elements can be configured to provide, for example, a massaging vibration or a poking action as the stimulus when selected.

In some embodiments, the selected one or more ones of the actuator elements can include all of the actuator elements. In some embodiments, the sleep adjustment device can be further configured to sense sleeping position of the user, and the one or more actuator elements can be selected based at least in part on the sensed sleeping position of the user. The one or more actuator elements can be selected to induce a change in sleeping position of the user from the sensed sleeping position to a new sleeping position having a lower likelihood of snoring. The new position having the lower likelihood of snoring can be determined based on a profile data associated with the user, with the profile data being included in or accessible by the control unit.

In some embodiments, the control unit can include a processor configured to build the profile data and/or to utilize the profile data. In some embodiments, such a processor can be further configured to execute an algorithm including an artificial intelligence algorithm.

In some embodiments, the sleep adjustment device can include a plurality of pressure sensors. In some embodiments, the plurality of pressure sensors can be co-located with respective ones of the plurality of actuator elements.

In some implementations, the present disclosure relates to a system for reducing snoring activity. The system includes non-transitory computer readable medium including data representative of a sleep profile of a person. The sleep profile is obtained from use of one or more patches, with each being configured to be secured to a skin of the person and sense snoring of the person, such that the sleep profile includes information about a snoring event during which the person is likely to snore. The system further includes a control unit in communication with the non-transitory computer readable medium and configured to generate a sleep plan for the person based at least in part on the information about the snoring event, with the sleep plan including a control signal. The system further includes a sleep adjustment device configured to receive the control signal and provide a stimulus to the person. The stimulus is selected to reduce the likelihood of snoring during a period associated with the snoring event.

In some embodiments, the control unit can be configured to generate the sleep plan without further use of a patch by the person. In some embodiments, the control unit can include a processor configured to generate the sleep plan utilizing an artificial intelligence algorithm.

In some embodiments, the control unit can be configured to generate the sleep profile. The sleep profile can be generated during a number of sleep periods such as night periods. In some embodiments, a new patch can be used for each sleep period, or a given patch can be for more than one sleep period.

In some embodiments, the number of sleep periods can be a fixed number of sleep periods. In some embodiments, the number of sleep periods can be determined by the control unit based at least in part on a confidence level of the information about the snoring event.

In some embodiments, the control unit can include a processor configured to generate the sleep profile utilizing an artificial intelligence algorithm.

In some embodiments, either or both of the sleep profile and sleep plan for the person can be based on information representative of likelihood of another person being disturbed by snoring of the person.

According to a number of implementations, the present disclosure relates to a patch that includes a wearable component configured to allow the patch to be worn on a user, and a sensor component implemented relative to the wearable component. The sensor component includes a transducer configured to generate an electrical signal based on a snoring sound.

In some embodiments, the wearable component can include a patch substrate configured to allow the patch to be worn on a skin of the user. The sensor component can be implemented at least partially within the patch substrate. The patch substrate can include an application surface configured to engage the skin of the user, and an open surface opposite from the application surface.

In some embodiments, the transducer can include a microphone. In some embodiments, the microphone can be implemented to receive the snoring sound incident on one of the application surface and the exposed surface. In some embodiments, the incident snoring sound can include a sound energy transmitted through air or a sound energy transmitted through a body of the user.

In some embodiments, the patch can further include an additional microphone implemented to receive a sound incident on the other of the application surface and the exposed surface. Either or both of the sound incident on the application surface and the sound incident on the exposed surface can include the snoring sound.

In some embodiments, the patch can further include an electronic circuit configured to process the electrical signal. The electronic circuit can be configured to identify a source of the snoring sound. The electronic circuit can be configured to perform the identification of the source of the snoring sound based on a signal processing algorithm. The electronic circuit can be configured to perform the identification of the source of the snoring sound based on one or more of intensity, frequency, and pattern associated with the snoring sound. The user can be the source of the snoring sound.

In some embodiments, the electronic circuit can be configured to route the electrical signal to a device external to the wearable patch.

In some embodiments, the patch can further include a communication component configured to communicate information associated with the electrical signal to a device external to the patch. The communication component can include a transmitter configured to generate a transmit signal based on the information associated with the electrical signal. The communication component can further include an antenna to support a wireless transmission of the transmit signal.

In some embodiments, the patch can further include an alert transducer configured to generate an alert stimulus for the user based on the electrical signal. The alert stimulus generated by the alert transducer can include a vibration signal or a low-intensity sound signal.

In some embodiments, the wearable component can be configured to allow the patch to be secured to the user with an adhesive layer. In some embodiments, the wearable component can be configured to allow the patch to be secured to a pierced body portion of the user. Such a wearable component can be configured to be worn as a body piercing device on a nose or an earlobe.

In some teachings, the present disclosure relates to a method for reducing snoring activity. The method includes sensing, with a transducer of a patch secured to a skin of a user, a snoring sound. The method further includes generating an electrical signal based on the snoring sound.

In some embodiments, the method can further include processing the electrical signal to identify a source of the snoring sound.

In some embodiments, the method can further include transmitting information associated with the electrical signal to a device external to the patch.

In some implementations, the present disclosure relates to a kit for reducing snoring activity. The kit includes a plurality of patches implemented in a packaged format, with each patch including a wearable component configured to allow the patch to be worn on a user, and a sensor component implemented relative to the wearable component. The sensor component includes a transducer configured to generate an electrical signal based on a snoring sound. The kit further includes a printed instruction configured to facilitate use of the patch.

For purposes of summarizing the disclosure, certain aspects, advantages and novel features of the inventions have been described herein. It is to be understood that not necessarily all such advantages may be achieved in accordance with any particular embodiment of the invention. Thus, the invention may be embodied or carried out in a manner that achieves or optimizes one advantage or group of advantages as taught herein without necessarily achieving other advantages as may be taught or suggested herein.

### BRIEF DESCRIPTION OF THE DRAWINGS

FIG. 1A depicts a non-invasive patch device that can be worn by a person during activities such as sleep.

FIG. 1B shows an example shape that can be implemented for the patch device of FIG. 1A with an adhesive functionality.

FIG. 1C shows another example shape that can be implemented for the patch device of FIG. 1A with an adhesive functionality.

FIG. 1D shows that in some embodiments, a device having one or more features as described herein can be configured to be secured to a body part such as a node or an ear lobe.

FIG. 1E shows that in some embodiments, a personal adornment item can be configured to provide one or more functionalities as described herein.

FIG. 1F shows that in some embodiments, the personal adornment item of FIG. 1E can be configured to be worn by a user in a pierced manner.

FIG. 2 shows a process that can be implemented by a system that includes one or more devices of FIGS. 1A-1F.

FIG. 3 shows that in some embodiments, a non-invasive patch having one or more features as described herein can have a generally rectangular shape.

FIG. 4 shows that in some embodiments, a non-invasive patch having one or more features as described herein can have an elliptical shape such as a circular shape.

FIG. 5A shows a person sleeping on a bed while wearing one or more devices in communication with a control unit to provide functionalities as described herein.

FIG. 5B shows an example of the control unit of FIG. 5A.

FIG. 5C shows a block diagram of example functionalities that can be provided by the control unit of FIG. 5B.

FIG. 6 depicts a block diagram of a system that can include a wearable device and be configured to perform one or more processes associated with FIG. 2.

FIG. 7 shows a system that can be a more specific example of the system of FIG. 6.

FIG. 8A shows an example bed system that includes one or more sleep adjustment devices in communication with one or more control units.

FIG. 8B shows that in some embodiments, the sleep adjustment device of FIG. 8A can include an articulating bed frame.

FIG. 8C shows that in some embodiments, a mattress having a sleep adjustment functionality as described herein can be configured to be used with a fixed frame.

FIGS. 8D-1 and 8D-2 show that in some embodiments, a sleep adjustment device having one or more features as described herein can include a sensing component configured to sense position of a sleeping person.

FIG. 8E shows that in some embodiments, a mattress can be configured to provide the sleep adjustment functionality of FIGS. 8D-1 and 8D-2 for each of a plurality of sleeping persons.

FIG. 8F shows that in some embodiments, a sleep adjustment device having one or more features as described herein can include a massage component configured to induce a change in sleeping position of a person.

FIG. 8G shows an example of sleeping position sensing functionality that can be implemented with a patch having one or more features as described herein.

FIGS. 8H-1 and 8H-2 show an example of how the massage component of FIG. 8F can induce a change in sleeping position.

FIG. 8I shows an example of an actuator device that can be utilized for the massage component of Figured 8F, 8H-1 and 8H-2.

FIG. 8J shows an example of user-specific calibration that can be implemented for a system as described herein.

FIG. 8K shows an example of the system associated with FIG. 8J can be configured to utilize the user-specific calibration information to reduce snoring of the user.

FIG. 8L shows an example of adaptive calibration and control functionality that can be implemented for a system as described herein.

FIG. 8M show that in some embodiments, a system as described herein can be configured to provide user-specific snore reduction functionality for each of a plurality of persons sleeping in close proximity to each other.

FIG. 9 shows that in some embodiments, a patch can include a patch substrate configured to support a number of components including a snoring sensor.

FIG. 10 depicts an example side sectional view of the patch of FIG. 9.

FIG. 11A shows that in some embodiments, a patch can include a microphone implemented to sense sound incident on an outer side of a patch substrate.

FIG. 11B shows that in some embodiments, a patch can include a microphone implemented to sense sound incident on an application side of a patch substrate.

FIG. 11C shows that in some embodiments, a patch can include a microphone implemented to sense sound incident on an outer side of a patch substrate, and a microphone implemented to sense sound incident on an application side of the patch substrate.

FIG. 12A shows a snoring sound sensing configuration that can be achieved with the patch of FIG. 11A.

FIG. 12B shows a snoring sound sensing configuration that can be achieved with the patch of FIG. 11B.

FIG. 12C shows a snoring sound sensing configuration that can be achieved with the patch of FIG. 11C.

FIG. 13 shows that in some embodiments, a patch can include a signal processing circuit configured to process a signal obtained by a snoring sensor.

FIG. 14 shows that in some embodiments, a patch can include a signal routing circuit configured to route a signal obtained by a snoring sensor, to a control unit associated with a separate device.

FIG. 15 shows a process that can be implemented to identify a snoring sound, by a patch having one or more features as described herein.

FIG. 16 shows a process that can be implemented to identify a user associated with snoring sound, by a patch having one or more features as described herein.

FIG. 17 depicts an example sleeping arrangement where one or more patches having one or more features as described herein can be utilized.

FIG. 18 depicts another example sleeping arrangement where one or more patches having one or more features as described herein can be utilized.

FIG. 19 shows an example of how a patch having one or more features as described herein can be utilized for the sleeping arrangement of FIG. 17.

FIG. 20 shows an example process that can be implemented for the configuration of FIG. 19.

FIG. 21 shows an example of how a patch having one or more features as described herein can be utilized for the sleeping arrangement of FIG. 18.

FIG. 22 shows an example process that can be implemented for the configuration of FIG. 21.

FIG. 23A shows an example sleeping arrangement in which each of two snoring sleepers wears a respective patch, and each patch communicates with a respective dedicated control unit.

FIG. 23B shows an example sleeping arrangement in which each of two snoring sleepers wears a respective patch, and both patches communicate with a common control unit.

FIG. 23C shows an example sleeping arrangement in which each of two snoring sleepers wears a respective patch, with each patch communicating with a respective control unit, and the control units communicating with each other.

FIG. 24 shows a process that can be implemented for the example configurations of FIGS. 23A-23C.

FIG. 25 shows an example configuration where a patch is associated with a personalized profile implemented in or accessible by a control unit.

FIG. 26 shows that in some embodiments, a control unit can include or have access to a plurality of personalized profiles associated with respective patches for use with different users.

FIG. 27 shows a process that can be implemented for the example configurations of FIGS. 25 and 26.

FIG. 28 shows a process that can be implemented to actuate a device or mechanism to provide a stimulus to a sleeping user wearing a patch.

FIG. 29 depicts a block diagram of a bed having first and second sleeping portions.

FIG. 30A depicts a side view of the bed of FIG. 29, where both mattresses are in a generally flat configuration.

FIG. 30B depicts a side view of the bed of FIG. 29, where one mattress can be adjusted to reduce or eliminate snoring of the person sleeping thereon, while the other mattress remains unchanged.

FIG. 31 shows an example where an activation of snoring-reducing stimulus can be provided by a device that may or may not be an integral part of a bed.

FIG. 32 shows that in some embodiments, a patch having a snoring sensor can also include a stimulus providing component.

FIG. 33 shows that in some embodiments, one or more patches having one or more features as described herein can be in a packaged format for use by a user.

FIG. 34A shows an example of a packaged format having a support sheet with a plurality of patches secured thereto.

FIG. 34B shows an example configuration of a patch that can provide one or more functionalities as described herein, and be provided in a packaged format.

FIG. 34C shows a more specific example of the packaged format of FIG. 34A.

FIG. 35 shows an enlarged side sectional view of an example support sheet that can be utilized to hold a plurality of patches in a packaged format.

FIG. 36 shows a process that can be implemented in a patch to allow use of the patch as described herein.

FIG. 37 shows a process that can be implemented in a controller to allow use of a corresponding patch.

FIG. 38 depicts a block diagram of a system that can include a patch having one or more features as described herein in communication with an external control unit.

FIG. 39 depicts a block diagram of a control unit that can be utilized as the control unit of the system of FIG. 38.

FIG. 40 shows an example of a communication functionality that can be implemented for a system having one or more features as described herein.

FIG. 41 shows another example of a communication functionality that can be implemented for a system having one or more features as described herein.

FIG. 42 shows that in some embodiments, a communication component of a patch can be configured to provide a wireless communication functionality with an external device.

FIG. 43 shows that in some embodiments, a communication component of a patch can be configured to provide a wired communication functionality with an external device.

FIG. 44 shows a system that can be formed with one or more patches as described herein, and an external device.

FIG. 45 shows that in some embodiments, the system of FIG. 44 can include a plurality of patches that communicate with a common external device.

FIG. 46 shows that in some embodiments, the system of FIG. 44 can include a plurality of patches that can communicate with each other, and/or with an external device.

### DETAILED DESCRIPTION OF SOME EMBODIMENTS

The headings provided herein, if any, are for convenience only and do not necessarily affect the scope or meaning of the claimed invention.

Disclosed are examples related to systems, methods and devices for sensing snoring of a user during sleep. Once sensed, one or more actions can be implemented to reduce or stop the snoring, to thereby benefit the user and/or anyone else sleeping nearby.

As generally understood and experienced by many, snoring during sleep can negatively impact the quality of sleep, and may be an indicator of health issues. Snoring can also disturb another person sleeping, or attempting to sleep, nearby.

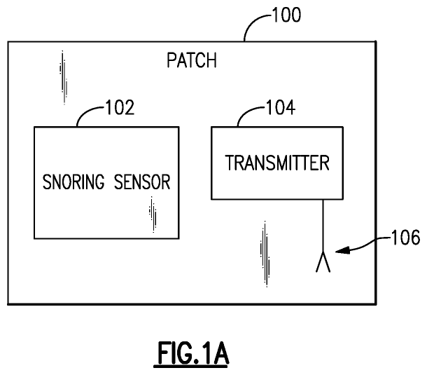

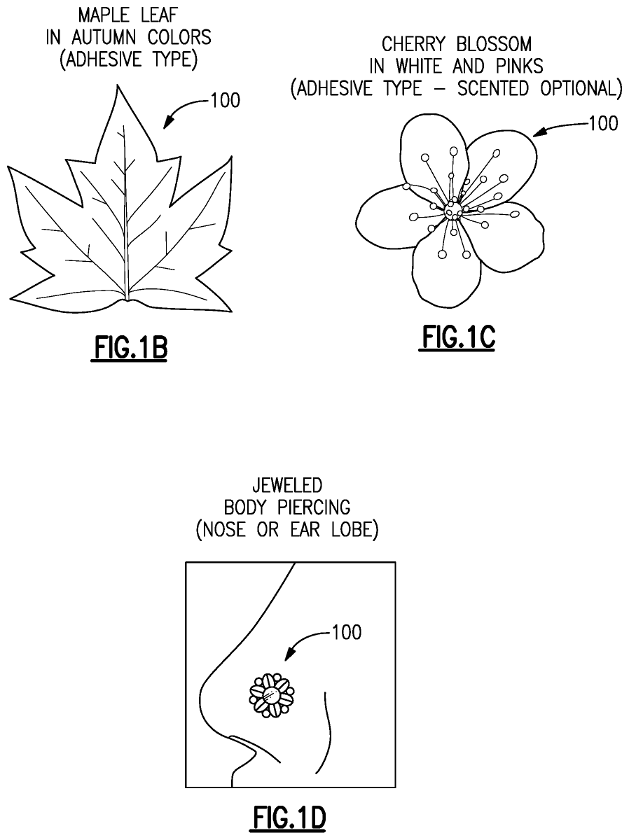

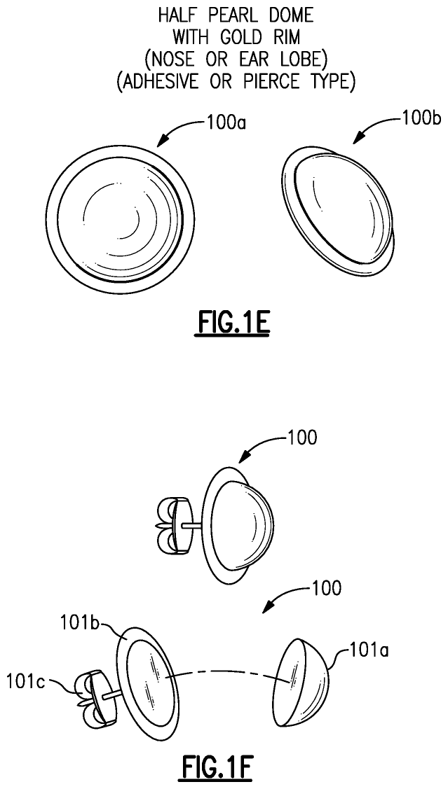

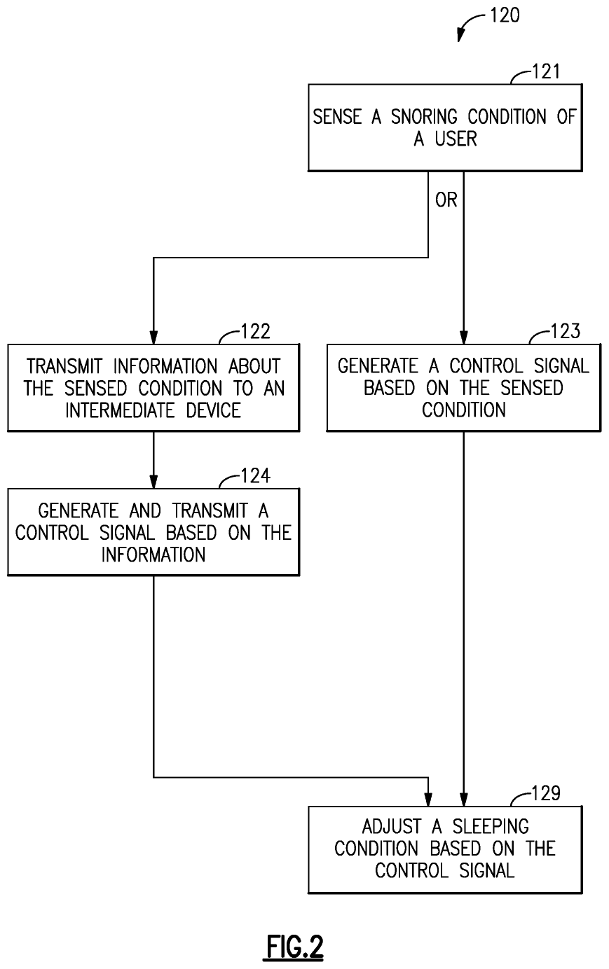

FIG. 1A depicts a patch device 100 that can be worn by a person during activities such as sleep. Although various examples are described herein in the context of sleep, it will be understood that one or more features of the present disclosure can also be utilized in other activities. For purposes of description, such a patch device may also be referred to herein as a patch, a non-invasive patch, a bio-patch, a non-invasive bio-patch, etc.

FIG. 1A shows that in some embodiments, the patch 100 can include a snoring sensor 102 and a transmitter 104 configured to transmit information about a snoring activity sensed by the sensor 102. In some embodiments, such transmission of information can be supported by an antenna 106 in communication with the transmitter 104.

In some embodiments, a patch having one or more features as described herein can be implemented with a form that appeals to some users. For example, FIG. 1B shows a patch 100 configured to be worn by a user with an adhesive component and having a nature-theme shaped form such as a maple leaf shape in autumn colors.

In another example, FIG. 1C shows a patch 100 configured to be worn by a user with an adhesive component and having a nature-theme shaped form such as a cherry blossom flower in white and pink colors. In some embodiments, such a patch can be configured to provide a corresponding scent (e.g., cherry blossom scent).

It will be understood that other shaped forms, including other nature-theme shaped forms, can be implemented for a patch having one or more features as described herein.

In yet another example, FIG. 1D shows a patch or a patch-based device 100 having one or more features as described herein and configured to be worn by a user as an item of jewelry. In the example of FIG. 1D, such an item of jewelry can be a body piercing type for wearing on a nose or an earlobe. Such an item of jewelry can include a shape, such as a flower shape, that appeals to a user.

FIG. 1E shows another example of a patch or a patch-based device 100 having one or more features as described herein and configured to be worn by a user as an item of jewelry. In the example of FIG. 1E, a top view (100a) and a perspective view (100b) of an item of jewelry are shown, and such an item of jewelry can be configured to provide one or more functionalities as described herein. In the example of FIG. 1E, such an item of jewelry can be configured to be worn on a body part such as a nose or an earlobe, and be secured thereto by an adhesive component or a piercing component.

FIG. 1F shows an example configuration where the patch or patch-based device 100 of FIG. 1 E is implemented as a body piercing type device. Such a device can include, for example, a jewelry portion 101a such as half pearl, and such a jewelry portion can be mounted to a piercing post portion 101b configured to provide, for example a gold (or similar) rim around the mounted half pearl 101a. The piercing post portion 101b can be secured to a user's body portion (e.g., a nose or an earlobe) with a nut portion 101c in engagement with a pin of the post portion 101b. In some embodiments, some or all of the foregoing components of the device 100 can be configured to provide one or more functionalities as described herein.

FIG. 2 shows a process 120 that can be implemented by a system that includes any one of the example patches or patch-based devices 100 of FIGS. 1A-1F. In block 121, snoring activity of a user can be sensed. In some embodiments, such sensing of the snoring activity can be achieved a patch or patch-based device as described herein. For the purpose of description herein, such a patch or patch-based device configured to sense snoring activity is referred to as a patch.

In FIG. 2, the foregoing sensed snoring activity can be utilized to adjust a sleeping condition in block 129. Examples of such adjustments are described herein in greater detail.

In some embodiments, a patch can be configured to communicate the sensed snoring activity information directly to a device that effectuates the adjustment of the sleeping condition. Accordingly, in block 123, a control signal can be generated based on the sensed snoring activity, and such a control signal can be utilized to adjust the sleeping condition (in block 129).

In some embodiments, an intermediate device can be utilized to provide control functionality between a patch and a device that effectuates the adjustment of the sleeping condition. In such a configuration, information about the sensed snoring activity can be transmitted by the patch to the intermediate device in block 122. Based on such sensed snoring activity information, a control signal can be generated and transmitted by the intermediate device in block 124. Such a control signal can be transmitted to the adjustment-effectuating-device to adjust the sleeping condition (in block 129).

In some embodiments, a system as described herein can be configured to provide either or both of the control functionalities of FIG. 2.

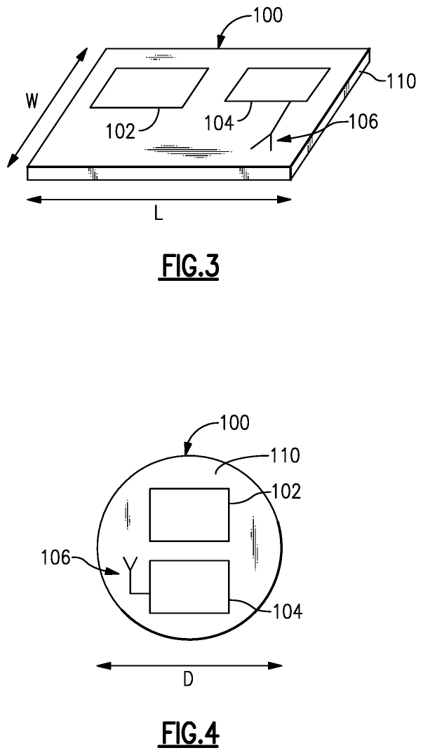

FIGS. 3 and 4 show non-limiting examples of how a patch 100 can be implemented in different form factors. For example, FIG. 3 shows that in some embodiments, a patch 100 can have a generally rectangular shape with a length L and a width W. Such dimensions can be selected to, for example, allow application of the patch 100 on an area of the user with sufficient space (e.g., on an arm).

In another example, FIG. 4 shows that in some embodiments, a patch 100 can have an elliptical shape, such as a circular shape, with a diameter D. Such a dimension can be selected to, for example, allow application of the patch 100 on a smaller area of the user that is more discreet.

It will be understood that a patch 100 having one or more features as described herein can be implemented with other shapes, including the example shapes of FIGS. 1B-1E.

In the examples of FIGS. 3 and 4, each patch 100 can include a patch substrate 110 configured to provide wearable functionality and to support a number of components. Examples related to such wearable functionality and support functionality can be found in U.S. Pat. No. 9,133,024 titled PERSONAL DIAGNOSTIC DEVICES INCLUDING RELATED METHODS AND SYSTEMS, which is expressly incorporated by reference in its entirely, and its disclosure is to be considered part of the specification of the present application.

In the examples of FIGS. 3 and 4, each patch 100 is shown to include a snoring sensor 102, a transmitter 104, and an antenna 106, similar to the example of FIG. 1A. In some embodiments, at least the transmitter portion of the patch 100 can be include an RFID (radio-frequency identification) circuitry configured to support transfer of information between the patch 100 and a device external to the patch 100.

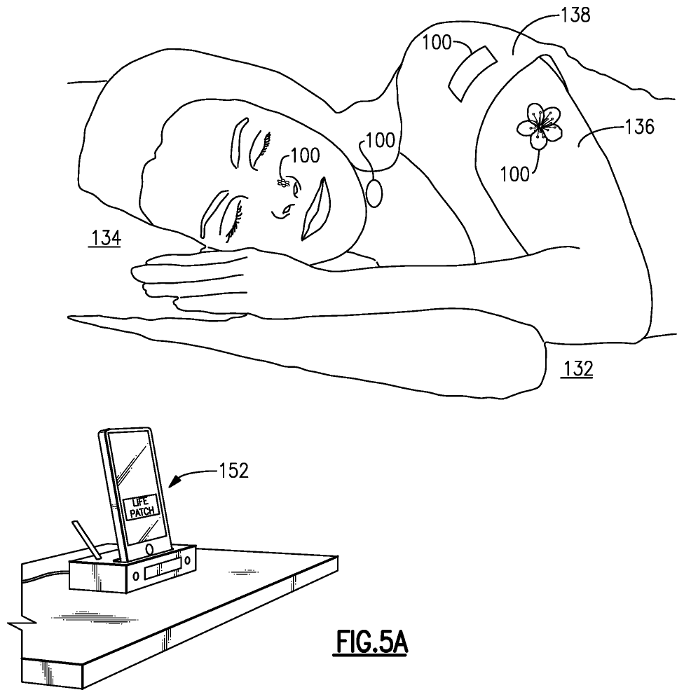

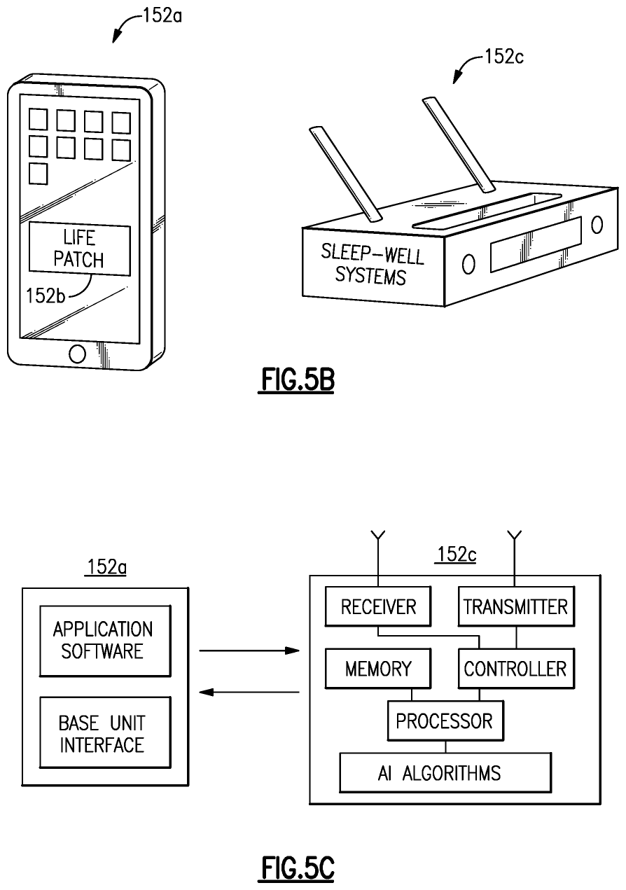

FIG. 5A shows a person 130 sleeping on a bed 132 with her head supported by a pillow 134. FIG. 5A further shows that such a sleeping person can wear a patch 100 having one or more features as described herein at one or more locations. For example, a patch 100 can be configured to be worn on a skin of an arm 136. In another example, a patch 100 can be configured to be worn on a surface of a clothing item 138. In yet another example, a patch 100 can be configured to be worn close to a sound source associated with the snoring activity (e.g., on the nose and/or on the throat area). In some embodiments, the patch(es) 100 worn by the user 130 can have form factors including the examples described herein.

It will be understood that a patch 100 having one or more features as described herein can be worn at other parts of a user. Such patch application locations can be based on, for example, whether a location is likely covered or exposed during sleep, whether a location is likely be impacted by various movements during sleep, whether a location provides an appropriate sensing condition, or some combination thereof.

In the example of FIG. 5A, a control unit 152 is shown to be nearby the sleeping person 130 so as to allow a patch 100 to communicate with the control unit 152. For example, the control unit 100 can be placed on a nightstand and be sufficiently close to the patch 100 to provide communication functionality therebetween. As described herein, such a control unit can communicate with the patch 100, and based on sensing of snoring of the person, generate and send a control signal to, for example, provide a stimulus to the snoring person to reduce or stop the snoring activity. Examples of such control functionality are described herein in greater detail.

FIG. 5B shows that in some embodiments, a control unit 152 having one or more features as described herein can include a portable device 152a such as a smart phone, and a base unit 152c configured to communicate with the portable device 152a and a patch (e.g., 100 in FIG. 5A). For example, the portable device 152a can include an application software (also referred to herein as an app) 152b configured to provide a user interface to, for example, enhance various functionalities that can be provided by the patch, the control unit 152, stimulus resulting from operation of the control unit, reduction or cessation of snoring based on the stimulus, or some combination thereof.

FIG. 5C shows an example of how the portable device 152a and the base unit 152c of FIG. 5B can be configured to provide control functionalities based on a patch and a sleep adjustment device. As shown in FIG. 5C, the portable device 152a and the base unit 152c can be configured to communicate with each other. Such communication can be achieved when the portable device 152a is docked on the base unit 152c, is separated from the base unit 152c, or some combination thereof.

Referring to the example of FIG. 5C, in some embodiments, the base unit 152c can be configured to include a receiver and an associated antenna; and such a receiver can be configured to receive a signal from a patch (e.g. 100 in FIG. 5A). In some embodiments, communication between the base unit 152c and the portable device 152a can be supported by the same receiver, by a separate receiver, or some combination thereof.

Referring to the example of FIG. 5C, in some embodiments, the base unit 152c can be configured to include a transmitter and an associated antenna that may or may not be the same as the foregoing receive antenna. Such a transmitter can be configured to support communication with the portable device 152a.

In embodiments where a patch is capable of receiving a signal from a control unit, such a control unit can include a transmitter to support such a communication link. In the context of the example of FIG. 5C, such transmission functionality can be provided by a patch-dedicated transmitter, a common transmitter capable of communicating with the patch and the portable device 152a, or some combination thereof.

Referring to the example of FIG. 5C, a controller component can be provided in the base unit 152c to control operation of the receiver and the transmitter. Such control functionality can be achieved by a processor that executes one or more algorithms (e.g., one or more artificial-intelligence (Al) algorithms). The processor can be provided with or have access to one or more memory components (e.g., a non-transitory computer readable medium) to facilitate the foregoing processing functionalities.

Referring to the example of FIG. 5C, in some embodiments, the portable device 152a can be configured to include an application software (also referred to herein as an app) to provide a user interface with a user (e.g., the sleeping person when awake, or someone else). The portable device 152a can be configured to further include an interface for communicating with the base unit 152c.

It will be understood that in some embodiments, some or all of various functionalities of the control unit 152 of FIG. 5A can be implemented in other configurations, including, for example, a single device, or multiple devices operatively configured to provide control functionalities.

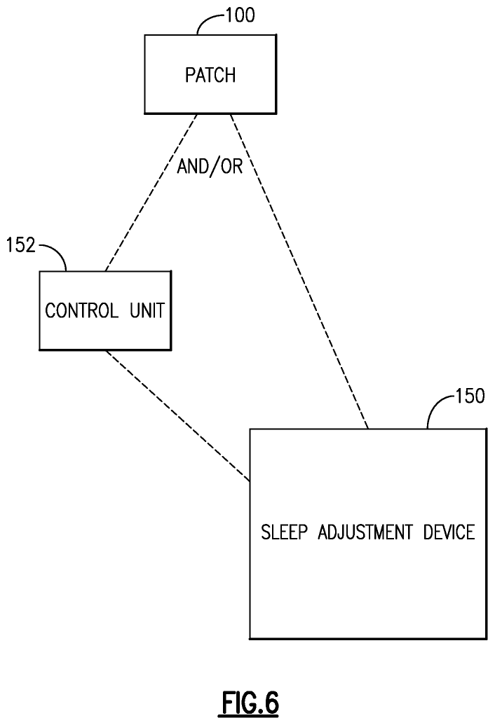

FIG. 6 depicts a block diagram of a system that can include a patch 100 having one or more features as described herein. In some embodiments, such a system can be configured to perform the process(es) described herein in reference to FIG. 2. FIG. 7 shows a configuration where the system of FIG. 6 includes a control unit 152 that provides one or more control functionalities between a patch 100 and a sleep adjustment device 150. In some embodiments, such a control unit can include, for example, the control unit 152 described in reference to FIGS. 5A-5C.

In the example of FIG. 6, the patch 100 can be configured to communicate directly with a device 150 having a sleeping condition adjustability. Examples related to such a sleep adjustment device are described herein. In some embodiments, either or both of the patch 100 and the sleep adjustment device 150 can include a control circuitry that allows control of the sleep adjustment device 150 based on the snoring activity sensed by the patch 100.

FIG. 6 further shows that in some embodiments, a separate control unit 152 can be provided to facilitate control of the sleep adjustment device 150 based on the snoring activity sensed by the patch 100. For example, the patch 100 and the control unit 152 can be configured to allow transfer of sensed information from the patch 100 to the control unit 152. Based on such sensed information, the control unit 152 can generate and provide to the sleep adjustment device 150 a control signal to actuate the sleep adjustment device 150.

In some embodiments, a system having one or more features as described herein can be configured to provide either or both of the foregoing control functionalities. It will be understood that a patch 100 having one or more features as described herein can be utilized to adjust a sleep adjustment device 150 with other control configurations.

FIG. 7 shows a system that can be a more specific example of the system of FIG. 6. In FIG. 7, a system can include a patch 100 having one or more features as described herein, and such a patch can be worn by a user 130, and be configured to communicate (depicted as 151) with a control unit 152. In some embodiments, such a communication can include information about sensed snoring activity of the user 130, and can be achieved in a wireless manner. In some embodiments, the control unit 152 can be, for example, part of a dedicated device, part of a personal electronic device such as a smartphone, or some combination thereof. In the context of the personal electronic device example, functionalities associated with the control unit 152 can be provided by and/or supported by, for example, an application program running on the personal electronic device.

In the example of FIG. 7, the control unit 152 can communicate (depicted as 153) with a sleep adjustment device 150 based on the sensed snoring activity of the user 130. In some embodiments, such a communication can be achieved in a wireless manner, and can include a control signal to actuate the sleep adjustment device 150. Examples of such a sleep adjustment device are described herein in greater detail.

In the example of FIG. 7, a patch is shown to be associated with a control unit and a corresponding sleep adjustment device. Thus, when a user wearing the patch snores in a detectable manner, the sleep adjustment device can be activated to reduce or stop the snoring of the user.

It is noted that if the user is sleeping by himself/herself, the snoring may not disturb others; accordingly, the system of FIG. 7 may not be particularly useful for reducing or eliminating such a disturbance of others with the snoring activity. However, there may be health benefits for reducing or eliminating the snoring activity of the user.

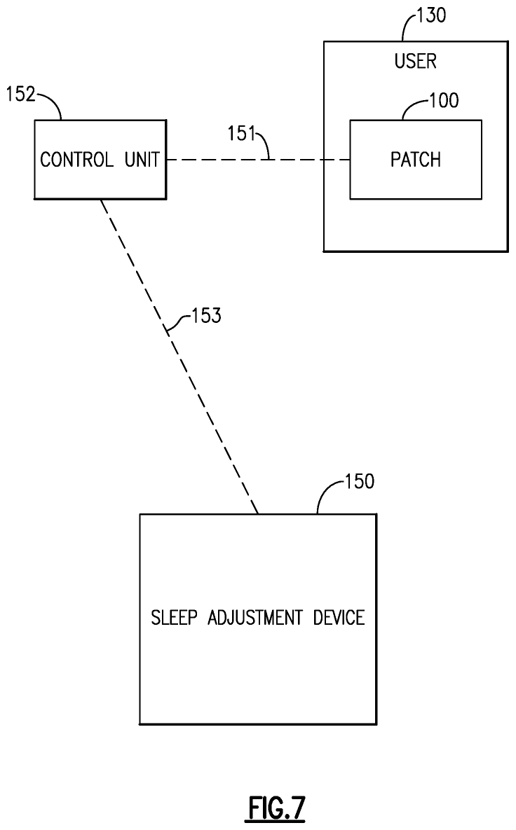

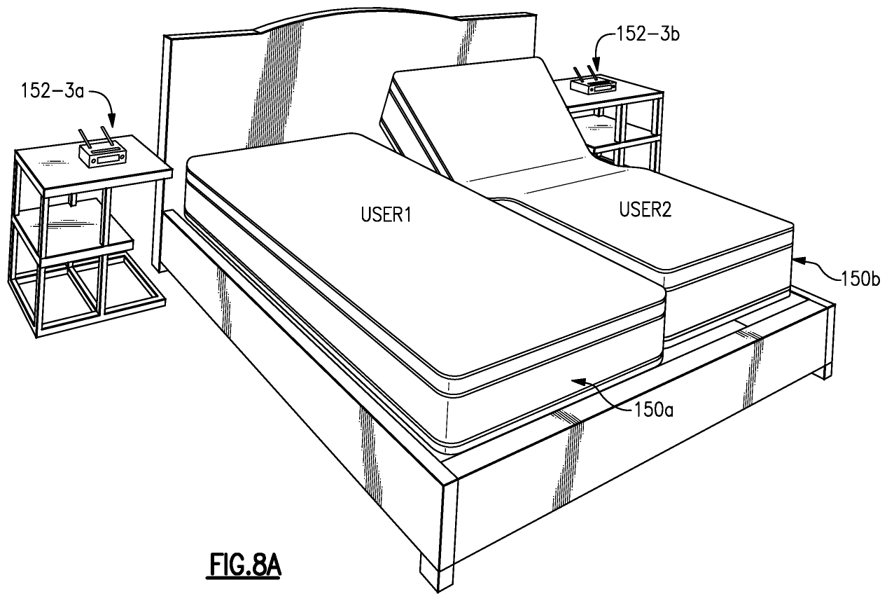

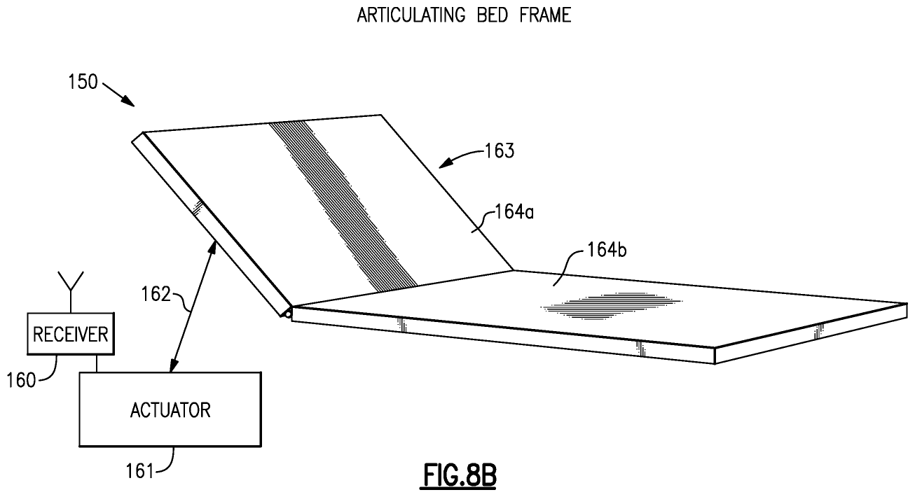

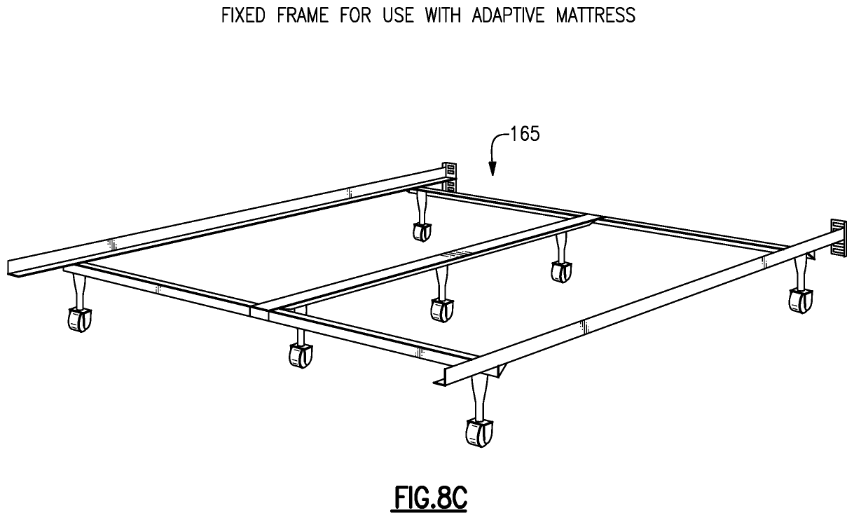

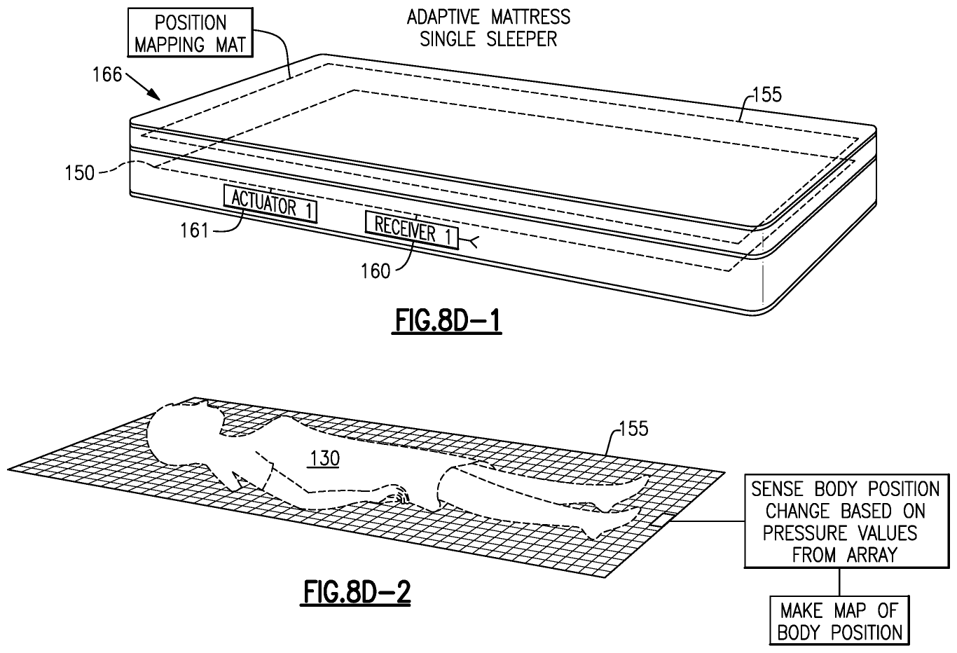

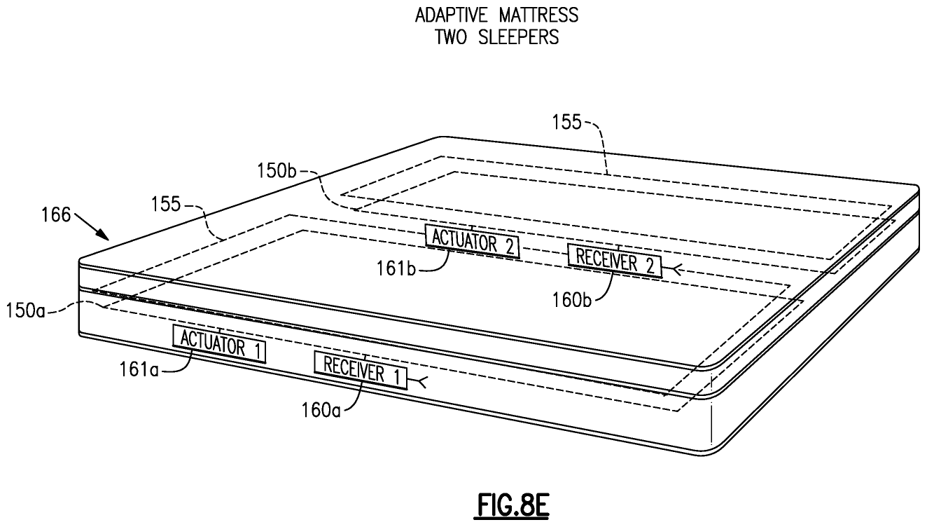

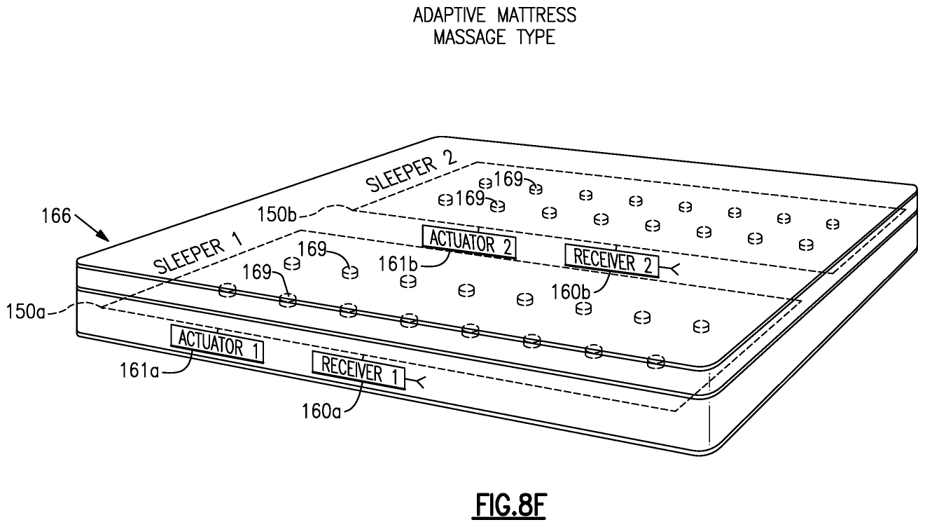

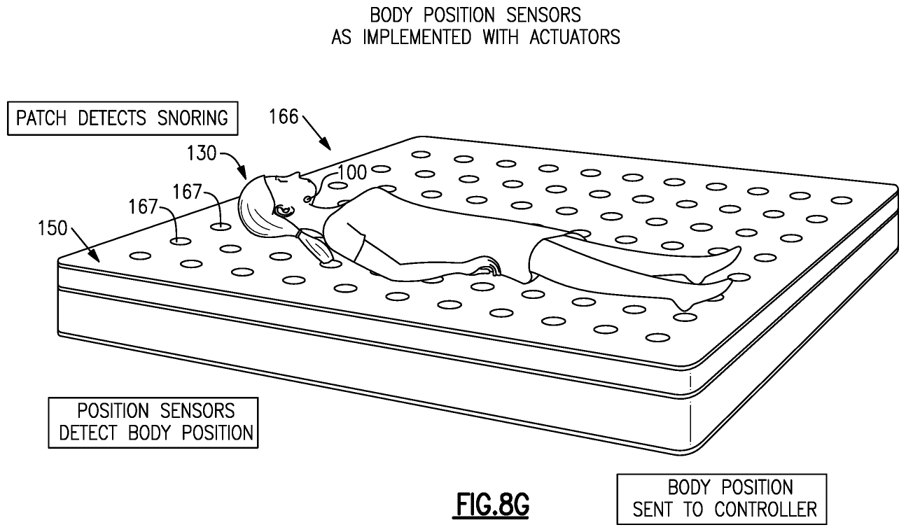

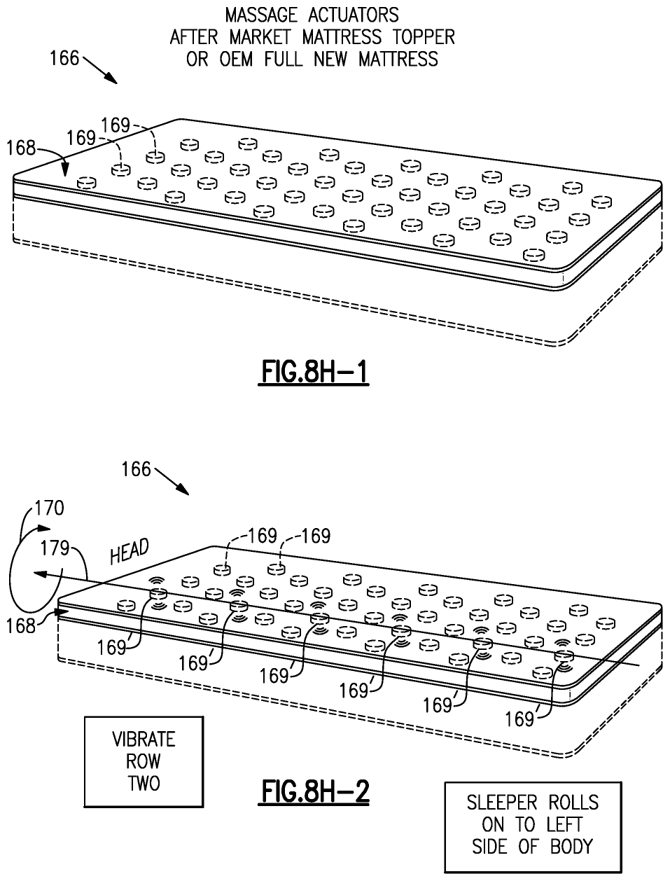

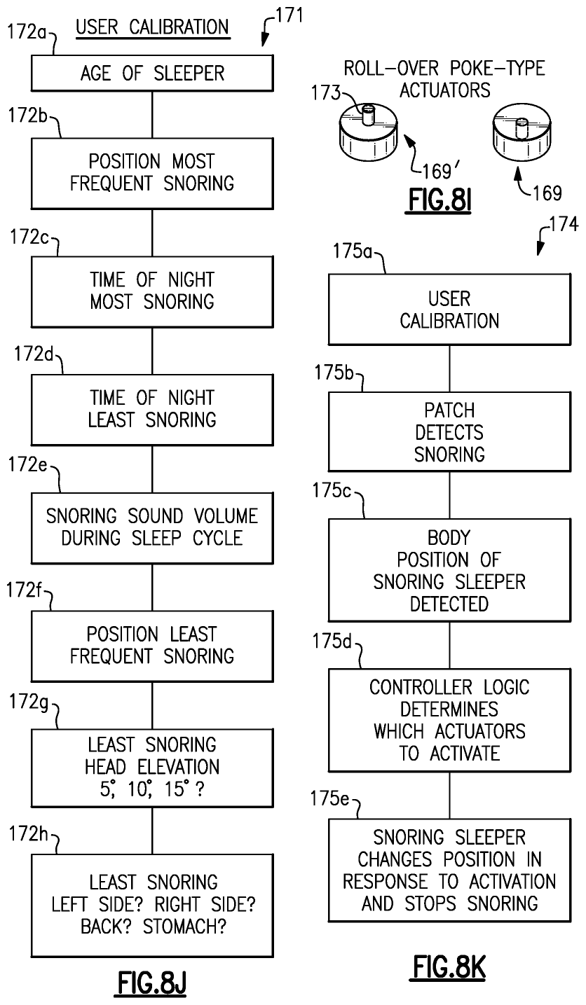

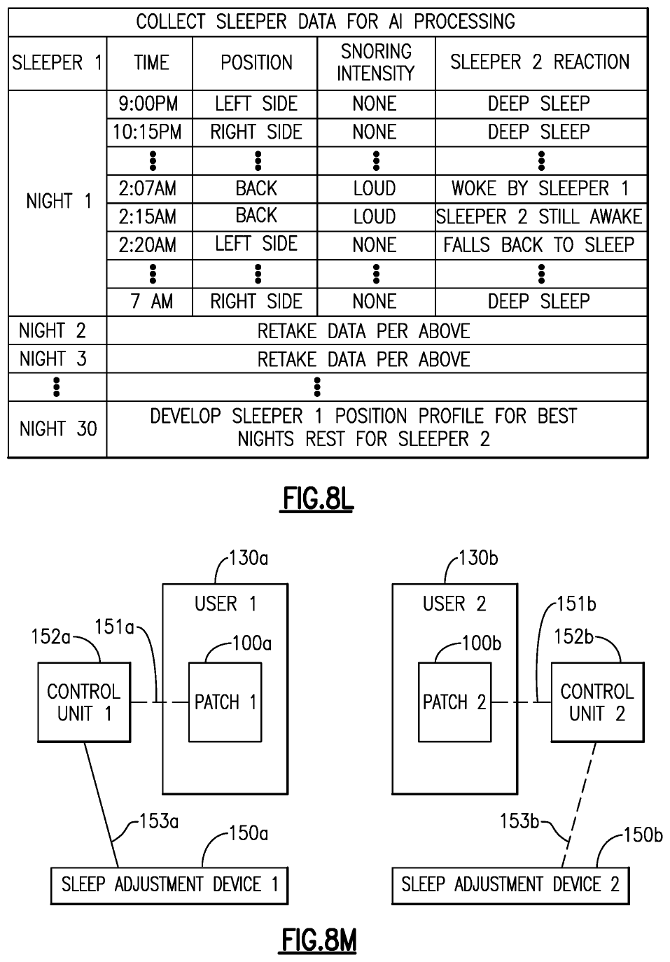

FIGS. 8A-8M show various examples related to the sleep adjustment device 150 of FIGS. 6 and 7. Such examples are based on an assumption that snoring activity of a user is detected by a patch, and based on such snoring detection, a control signal is generated by a control unit. As described herein, such a control signal can be utilized to adjust a sleeping condition provided to the user. In some embodiments, such adjustment of the sleeping condition can be actuated based on the detection of snoring, be actuated based on the detection of snoring and some user-specific information, be actuated based on the detection of snoring and another sensed information about the sleeping user, or some combination thereof.

In some embodiments, a snore-reduction system, such as the example of FIG. 6 or FIG. 7 can be implemented with an articulating bed (also referred to as an adjustable bed) or a fixed bed. FIGS. 8A and 8B show examples of sleep adjustment devices of snore-reduction systems in the context of an articulating bed, and FIGS. 8C-8H show examples of sleep adjustment devices of snore-reduction systems in the context of a fixed bed. However, it will be understood that one or more features described in one context (e.g., articulating bed or fixed bed) can also be implemented in the other context (e.g., fixed bed or articulating bed).

FIG. 8A shows a bed assembly having two separate articulating beds for two persons (User 1 and User 2). In some embodiments, each of such articulating beds can include a sleep adjustment device configured to communicate with a respective control unit and provide a stimulus to a respective sleeping person in response to a patch detecting snoring of the sleeping person. More particularly, the first articulating bed can include a first sleep adjustment device 150a in communication with a first control unit 152-3a. Such a first control unit can be in communication with a patch worn by User 1 when that person sleeps on the first articulating bed. Similarly, the second articulating bed can include a second sleep adjustment device 150b in communication with a second control unit 152-3b. Such a second control unit can be in communication with a patch worn by User 2 when that person sleeps on the second articulating bed.

FIG. 8B shows an articulating bed frame 163 that can be a part of each articulating bed of FIG. 8A. In some embodiments, such an articulating bed frame can be configured to operate with an actuator 161 (as indicated with arrow 162), in response to a control signal received by a receiver 160. Such a control signal can be provided by, for example, the respective control unit (152-3a or 152-3b) of the example of FIG. 8A. Accordingly, in some embodiments, the assembly of parts depicted in FIG. 8B can be a sleep adjustment device 150 for each of the two articulating beds of FIG. 8A.

In some embodiments, a sleep adjustment device, such as the sleep adjustment device(s) 150 of FIGS. 6 and 7, can be implemented to be a part of a mattress, as an additional device (e.g., an aftermarket device such as a mattress topper) configured to be used with an existing mattress, or some combination thereof. Accordingly, for the purpose of description, it will be understood that an adaptive mattress can be a mattress having built-in sleep adjustment functionality and/or an assembly of an existing mattress and an additional device (with a sleep adjustment functionality). It will also be understood that such an existing mattress may or may not have a built-in sleep adjustment functionality.

FIG. 8C shows a fixed frame 165 configured to support an adaptive mattress having one or more features as described herein. In some embodiments, such an adaptive mattress can be implemented in different sizes to support one or more persons thereon. Accordingly, the fixed frame 165 can be dimensioned and/or be adjustable to support different sized mattresses.

In some embodiments, an adaptive mattress can include a body position sensing functionality. As described herein, information obtained from such body position sensing functionality can be utilized with snore sensing functionality of a patch to, for example, provide a relationship, or an estimate thereof, between likelihood of snoring and sleeping body position. In some embodiments, with such a relationship information, a stimulus can be provided to a snoring person to induce a change in body position to a new position with a lower likelihood of snoring. Examples of such sleeping body position sensing functionality are described herein in greater detail.

FIGS. 8D-1 and 8D-2 show an example of how a body sensing functionality can be implemented in an adaptive mattress 166. In some embodiments, such an adaptive mattress can include a body position sensor 155 implemented as a position mapping mat to detect one or more body positions of a person sleeping thereon. Such a position mapping mat can be dimensioned and positioned on or in the adaptive mattress 166 so as to provide a sensing area that covers some of all of an area where the person's body parts are likely to engage the sleeping surface on the mattress. For example, and as shown in FIG. 8D-2, the sensing area of the position mapping mat 155 can be similar to the sleeping area of the mattress 166, and include an array of pressure sensing detectors.

As also shown in FIG. 8D-1, the pressure sensor array can allow sensing of the person's sleeping position based on pressure values obtained from the array to form a corresponding map of the sensed position. For example, if the person is sleeping on the back as shown, sensor readings from pressed sensors under the head, shoulder, arms, back and legs can be utilized to determine that the person is on the back with arms at the sides, and the legs straight. In another example, on-the-back position and on-the-stomach position can be distinguished by, for example, differences in pressure profiles obtained from the torso region. In yet another example, on-the-back/on-the-stomach position and on-the-side position can be distinguished by, for example, determining pressure readings indicative of one shoulder/upper arm (on-the-side) or both shoulders/upper arms (on-the-back)

In the example of FIG. 8D-1, the adaptive mattress 166 is shown to include the body position mapping mat 155 and a sleep adjustment device 150. As described herein, such a sleep adjustment device can be operated with an actuator 161 based on a control signal received through a receiver 160. In some embodiments, the sleep adjustment device 150 can be operated based on a control signal resulting from sensing of snoring by a patch worn by the sleeping person 130. In some embodiments, a control signal for operating the sleep adjustment device 150 can be further based on the sleeping person's body position sensed by the body position sensor 155. Examples of such body position based controlling of the sleep adjustment device are described herein in greater detail.

In some embodiments, the body position sensor 155 can be configured to communicate with the control unit to allow the control unit to combine information about body position and/or change in body position with information about snoring activity obtained from the patch. Such sensing and communication functionalities can be implemented in the same circuitry as the circuitry associated with the sleep adjustment device 150 (e.g., actuator 161 and receiver 160), in a separate circuitry, or some combination thereof.

FIG. 8E shows that in some embodiments, an adaptive mattress can be configured to support a plurality of sleeping persons (e.g., two persons). Such an adaptive mattress can include a sleep adjustment device and a body position sensor for each person. More particularly, an adaptive mattress 166 can include a first sleep adjustment device 150a and a corresponding body position sensor 155 provided for the first person, and a second sleep adjustment device 150b and a corresponding body position sensor 155 provided for the second person. The first adjustment device 150a can be controlled through a first receiver 160a and a first actuator 161a in a manner similar to the example of FIG. 8D-1. Similarly, the second adjustment device 150b can be controlled through a second receiver 160b and a second actuator 161b in a manner similar to the example of FIG. 8D-1. Each body position sensor can be configured to provide sensing and communication functionalities in a manner similar to the example of FIG. 18D-1.

FIG. 8F shows that in some embodiments, an adaptive mattress can include a sleep adjustment device having a plurality of stimulus-generating actuators (e.g., massage type actuators). Such stimulus-generating actuators, indicated as 169, are shown to be parts of each of a first sleep adjustment device 150a and a second sleep adjustment device 150b. In some embodiments, such stimulus-generating actuators can be actuated by a respective actuator circuitry (161a or 161b) in response to a control signal received through a respective receiver (160a or 160b).

In some embodiments, some or all of the stimulus-generating actuators 169 of FIG. 8F can be actuated based on a control signal resulting from detection of snoring by a patch worn by a sleeping person. In some embodiments, all of the stimulus-generating actuators 169 can be actuated to provide stimulus to any part of the body of the sleeping person, and such a stimulus may be sufficient to cease or reduce the snoring of the sleeping person. In some embodiments, one or more selected stimulus-generating actuators 169 can be actuated to provide stimulus to a selected part of the body of the sleeping person, and such a stimulus may reduce or stop the snoring of the sleeping person. In some embodiments, one or more selected stimulus-generating actuators 169 can be actuated to provide stimulus to induce a change in body position of the sleeping person, and such a change in body position may reduce or stop the snoring of the sleeping person. Various examples related to the stimulus-generating actuators 169 are described herein in greater detail.

In some embodiments, the foregoing functionality of reducing or stopping snoring with a plurality of stimulus-generating actuators 169 can be implemented such that the stimulus-generating actuators 169 are dedicated actuators configured to be actuated upon detection of snoring. In some embodiments, the foregoing functionality of reducing or stopping snoring with a plurality of stimulus-generating actuators 169 can be implemented such that the stimulus-generating actuators 169 also provide one or more other functionalities. For example, the stimulus-generating actuators 169 can be configured to provide massage for a person thereon, to some or all portions of the person's body in contact with the mattress.

As described in reference to FIGS. 8D and 8E, an adaptive mattress can include a body position sensing functionality and a sleep adjustment functionality. For the former functionality, FIG. 8F is an example where the sleep adjustment functionality can be implemented with an array of stimulus-generating actuators 169. For the latter functionality, FIG. 8D-2 is an example where an array of sensors such as pressure sensors can be utilized to determine a body position of a person sleeping thereon.

In some embodiments, an adaptive mattress can be configured so that at least some of body position sensing elements are implemented to be co-located, or sufficiently near, respective stimulus-generating actuator elements. Such a configuration can allow identification of a selected portion in a sensed body position and provide a stimulus to a selected portion of the body. It will be understood that the selected portion in the sensed body position may or may not be the same as the selected portion of the body where the stimulus is provided.

FIG. 8G shows an example of an adaptive mattress 166 with the foregoing feature where at least some of body position sensing elements are implemented to be co-located, or sufficiently near, respective stimulus-generating actuator elements. In the example of FIG. 8G, the adaptive mattress 166 is shown to include a sleep adjustment device 150 with an array of body position sensing elements 167. In some embodiments, each of such elements (167) can also be configured to provide a stimulus-generating functionality. Thus, a person 130 sleeping on the adaptive mattress 166 can have his/her body position sensed as described herein, and based on such body position information and detection of snoring by a patch 100, provide a stimulus to a selected portion of the sleeping person's body. As described herein, such a stimulus itself can reduce or cease snoring of the person, induce the sleeping person to a new body position where snoring is less likely, or some combination thereof.

FIGS. 8H-1 and 8h-2 show an example where an adaptive mattress 166 can be configured to induce a body position change upon detection of snoring by a patch worn by a user sleeping thereon. In the example of FIGS. 8H-1 and 8h-2 an array of stimulus-generating actuators 169 can be implemented as an aftermarket mattress topper or be a part of a mattress. In some embodiments, and as described herein, the stimulus-generating functionality provided by the actuators 169 can be co-located with body-position sensing elements (e.g., 167 in FIG. 8G). Accordingly, for the example of FIGS. 8H-1 and 8h-2, it will be assumed that position of the sleeping person is determined when the patch detects the snoring activity.

Referring to FIGS. 8H-1 and 8H-2, suppose that the person is sleeping on the back, roughly at the middle of the mattress 166, and the head is on the end as indicated. Accordingly, a line 179 that extends approximately parallel to and to the right side of the person's midline (e.g., along the spine) can represent, for example, a line extending through the right shoulder and the right leg, as sensed by the array of body-position sensing elements co-located with the actuator elements 169.

Upon sensing of snoring activity of the person, the control unit can determine that changing of sleeping position from the current position on the back to a new position on the left side can reduce or stop the snoring activity. Thus, an actuation signal can be provided to a selected group of actuator elements, and such actuated elements are indicated as 169′ in FIG. 8H-2 along the line 179. Stimulus provided by such selected actuated elements (e.g., along the second row corresponding to the right side of the person's midline) can induce the sleeping person to roll (indicated as 170) on to the left side of the body to reduce or stop the snoring activity.

FIG. 8I shows that in some embodiments, an actuator element, such as the actuator element 169 of FIGS. 8H-1 and 8H-2, can be configured to induce a body position change. For example, a poke-type actuator element can be configured to have a poking member 173 in a retracted position (indicated as 169) or an actuated position (indicated as 169′). The poking member 173 can be configured to extend and engage a respective portion of the sleeping person's body to induce a selected change in body position.

As described herein, a patch can be configured to detect a snoring activity of a user wearing the patch. When a system utilizes such a patch functionality, a stimulus can be provided to the user to reduce or stop the snoring activity.

In some embodiments, a system can be configured to form calibration data for a user based on snoring detection provided by a patch, and such calibration data can be utilized to enhance sleep quality for the user and/or another person. For the purpose of description, such a calibration data associated with the user may also be referred to as, for example, a snoring profile, a sleep profile, a user profile, or simply a profile.

In some embodiments, a system having the foregoing calibration data can be configured to provide improved quality of sleep for the snoring person corresponding to the calibration data, with or without further use of a patch. In sleeping arrangements involving a plurality of persons, a system having the foregoing calibration data can be configured to provide improved quality of sleep for the snoring person corresponding to the calibration data and/or another person, with or without further use of a patch by the snoring person.

FIGS. 8J-8L show various examples related to the foregoing user calibration data and how such calibration data can be utilized to enhance sleep quality of one or more persons. More particularly, FIG. 8J shows an example calibration data or user profile 171 that can be formed by a system utilizing a patch having one or more features as described herein. In some embodiments, such calibration data can be formed by collection of information over a calibration period involving a plurality of days for a user. It will be understood that the user can utilize one patch for the calibration period, a new patch for each day, or some combination thereof. It will also be understood that while sleeping is assumed to occur during night time, one or more features of the present disclosure can also be implemented for a user sleeping during day time or any other time.

Referring to FIG. 8J, the example calibration data 171 can include some or all of age of the user (172a), sleeping position associated with most frequent snoring (172b), time of night associated with most snoring (172c), time of night associated with least snoring (172d), snoring sound volume during sleep cycle (172e), sleeping position associated with least frequent snoring (172f), head elevation (e.g., elevation angle) associated with least snoring (172g), and body position (e.g., left side, right side, back, stomach) associated with least snoring (172h).

FIG. 8K shows an example where the user calibration data 171 of FIG. 8J can be utilized by a system. In block 175a, user calibration data associated with a sleeping person can be accessed by, for example, a control unit of the system. In block 175b, a patch worn by the sleeping person can detect snoring. In block 175c, body position of the snoring person can be detected (e.g., by an adaptive mattress). In block 175d, the control unit can determine a new body position based on the detected body position from block 175c and the user calibration data (171) and generate a control signal for inducing such a change in body position. In block 175e, the snoring person can be induced to change body position to the new body position in response to activation of stimulus provide to the snoring person (e.g., stimulus provided by one or more selected poke-type of massage-type actuators based on the control signal), to thereby stop or reduce the snoring.

FIG. 8K is an example where a system provides a stimulus to a sleeping person to reduce or stop snoring by that person, based on snoring detection provided by a patch worn by the person. In some embodiments, a system can be configured to provide a predictive configuration of a bed, such as an adaptive mattress, for a user based on a his/her calibration data but not relying on further snoring detection by a patch. Such a system can then allow the user and/or another person to obtain quality sleep without having the user to continue to wear a patch.

For example, FIG. 8L shows an example where data can be collected for a first person (Sleeper 1) over a number of nights, and based on such data, form a sleeping position profile to be implemented for the first person without relying on further use of a patch by the first person. Such sleeping position profile can be configured to provide enhanced sleep quality for Sleeper 1 and/or another person (Sleeper 2) sharing the same bed. In some embodiments, a system can include a control component with artificial intelligence (AI) processing capability, predictive processing capability, etc. to achieve at least some of the foregoing functionalities.

Referring to the example of FIG. 8L, in some embodiments, processing of sleeper data collection and generation of predictive sleeping position profile can be achieved by an AI computing device. More particularly, sleeper data collection can be implemented over a number of nights while Sleeper 1 wears a patch. In some embodiments, such number of nights can be a predetermined number, be based on a running evaluation of collected data, or some combination thereof. For example, Sleeper 1 may have a very consistent sleeping pattern, including snoring pattern. In such a situation, the AI processing can determine, in a running evaluation, that reliable data can be obtained during a small number of nights to allow development of a reliable sleeping position profile to be implemented for Sleeper 1.

In the example of FIG. 8L, a detailed listing of events of Sleeper 1 is shown, as well as responses of Sleeper 2 to such events. Each event is shown to be provided with a time stamp to allow correlation between the event of Sleeper 1 and the corresponding response of Sleeper 2. Each event is also shown to include a sleeping position of Sleeper 1, and intensity of snoring of Sleeper 1. The sleeping position of Sleeper 1 can be determined by utilizing, for example, an adaptive mattress (e.g., 166 in FIGS. 8D and 8G). The intensity of snoring can be determined by utilizing a patch as described herein. The reaction of Sleeper 2 to each event of Sleeper 1 can be determined by, for example, a wearable device configured to provide sleep information.

In the example of FIG. 8L, the first event of the first night is shown to be at 9:00 PM, where left side sleeping position is detected, and no-snoring is detected for Sleeper 1; and Sleeper 2 is shown to be in a deep sleep. At 10:15 PM, right side sleeping position is detected, and no-snoring is detected for Sleeper 1; and Sleeper 2 is shown to be in a deep sleep. Further events can be triggered periodically at fixed time intervals, be triggered by a change in sleeping position or snoring intensity, or some combination thereof.

Thus, continuing with example events, at 2:07 AM, back sleeping position is detected, and loud-snoring is detected for Sleeper 1; and Sleeper 2 is shown to be awake, likely be the loud-snoring of Sleeper 1. At 2:15 AM, back sleeping position is detected, and loud-snoring is detected for Sleeper 1; and Sleeper 2 is shown to be still awake. At 2:20 AM, left side sleeping position is detected, and no-snoring is detected for Sleeper 1; and Sleeper 2 is shown to have fallen back to sleep.

Events can continue to be recorded until, for example, a pre-set time or when Sleeper 1 is detected to get up from the bed. In the example of FIG. 8L, the last event is shown to be at 7:00 AM, where right side sleeping position is detected, and no-snoring is detected for Sleeper 1; and Sleeper 2 is shown to be in a deep sleep.

As shown in the example of FIG. 8L, events can be recorded for a number of subsequent nights, and based on data from such recorded events, a sleeping position profile can be generated for Sleeper 1. Such a generated profile can be implemented for Sleeper 1 to, for example, minimize or reduce snoring at predictively selected time(s) by Sleeper 1. Such a generated profile can be based on the sleeping pattern of Sleeper 1, on the sleeping pattern of Sleeper 2, or some combination thereof.

For example, suppose that sleeping pattern of Sleeper 2 is easily affected by snoring of Sleeper 1, and resulting disruption of sleep of Sleeper 2 has a significant negative effect on Sleeper 2 on the following day. In such an example situation, the generated profile for Sleeper 1 can be configured to provide an enhances sleep quality for Sleeper 2. For example, at a number of minutes (e.g., 5 minutes) before an average time of onset of loud snoring (e.g., around 2:07 AM), an adaptive mattress can be actuated to induce or maintain a sleeping position of Sleeper 1 of left side or right side, and such a sleeping position can be maintained up to a number of minutes (e.g., 5 minutes) past an average time of end of loud snoring (e.g., around 2:15 AM). Such a configuration of the adaptive mattress controlled by the generated profile can allow Sleeper 2 to have a higher-quality sleep by not being awakened by loud snoring of Sleeper 1.

In the example of FIG. 8L, it is assumed that one person (Sleeper 1) snores and the other person (Sleeper 2) does not snore. Accordingly, Sleeper 2 would not wear a patch for the purpose of sensing his/her own snoring. In some situations, however, both sleepers may snore; and in such situations, it may be desirable to have both persons wear respective patches to benefit as described herein.

For example, FIG. 8M shows an example arrangement where two users sleep in close proximity to each other. In such an arrangement, a first user 130a can wear a patch 100a, and a second user 130b can wear a patch 100b. In some embodiments, each of the patches 100a, 100b can have associated with it a control unit (152a or 152b) and a sleep adjustment device (150a or 150b), similar to the example of FIG. 7.

In the example of FIG. 8M, each patch (100a or 100b) can have associated with it a separate control unit (152a or 152b). It will be understood that in some embodiments, both of the patches 100a, 100b can be controlled by a common control unit. Examples related to such control functionality are described herein in greater detail.

In some embodiments, the sleep adjustment devices 150a, 150b can be parts of the same bed or bed assembly having one or more adaptive mattresses as described herein. For example, FIGS. 8A, 8E, 8F show examples where the corresponding beds are configured to accommodate two sleepers.

FIG. 9 depicts a patch 100 having one or more features as described herein. FIG. 9 shows that in some embodiments, and as described herein in reference to FIGS. 3 and 4, a patch 100 can include a patch substrate 110 configured to support a number of components.

For example, a snoring sensor 102 can be implemented on and/or within such a patch substrate 110. Such a snoring sensor can include, for example, an electroacoustic transducer 112 and electronics 114 configured to support operation of the electroacoustic transducer 112. The patch 100 can further include a communication component 103 configured to support one or more communication functionalities associated with the patch 100. In some embodiments, such a communication component can communicate with the electronics 114 so as to, for example, transmit information about snoring activity sensed by the electroacoustic transducer 112.

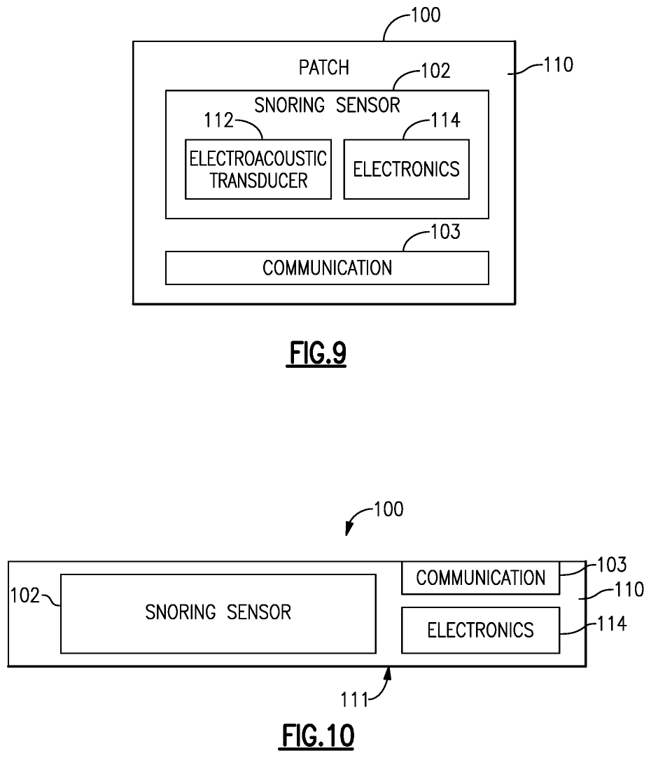

FIG. 10 depicts an example side sectional view of the patch 100 of FIG. 9. In the example of FIG. 10, a patch 100 can be configured so that a snoring sensor 102 is implemented to be at least partially within a patch substrate 110. For the purpose of description, the patch substrate 110 can include an application surface 111 configured to allow application of the patch 100 on a user (e.g., on a skin of the user). In such a configuration, the opposite side of the patch substrate 110 can be an outer side that faces away from the application surface such as skin.

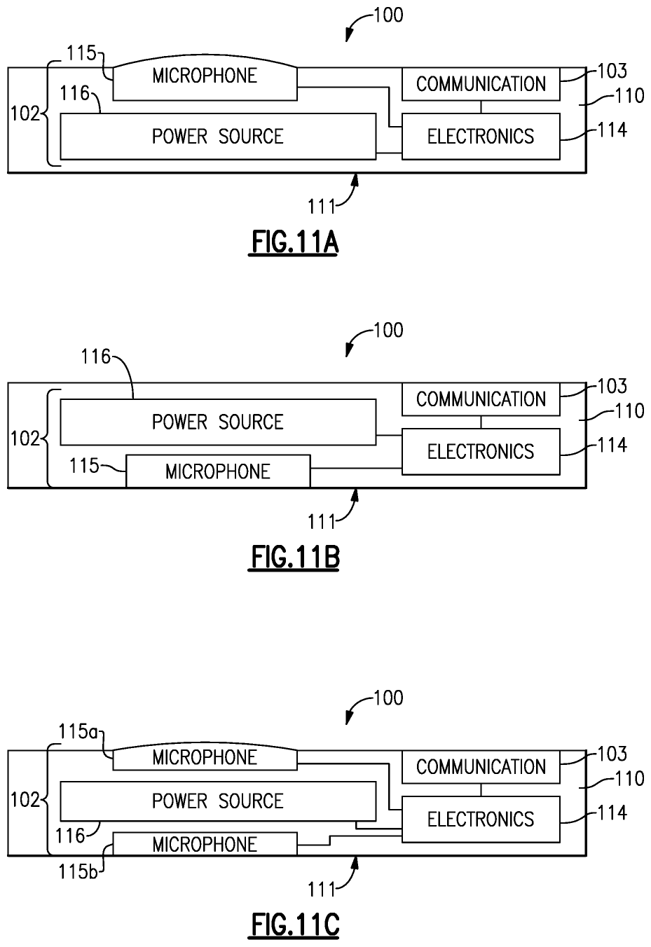

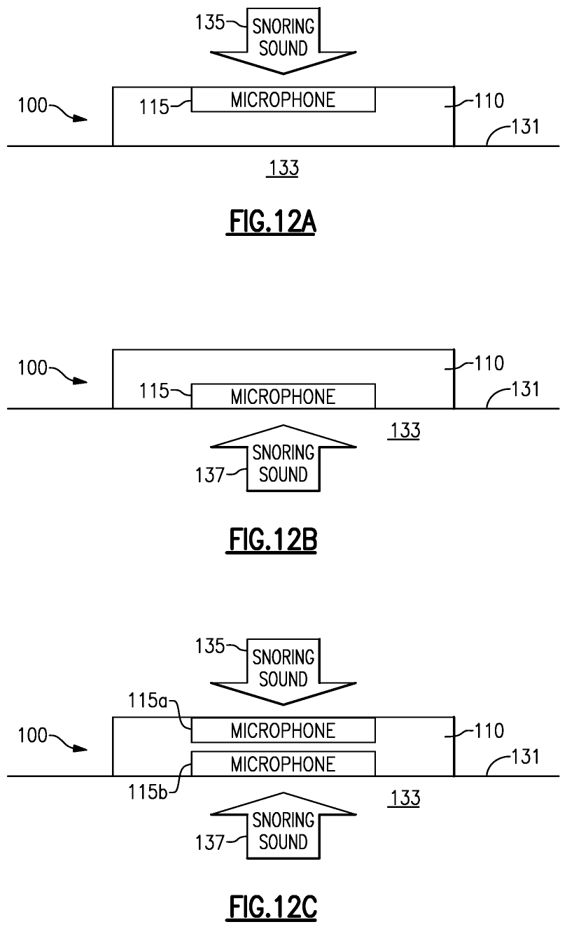

Referring to FIG. 10, the snoring sensor 102 can be implemented so as to allow sensing of sound from either or both of the application surface side (e.g., sound arriving through tissue) and the outer side (e.g., sound arriving through air). Examples of such sound sensing configurations are described herein in reference to FIGS. 11 and 12.

FIG. 10 shows that in some embodiments, some or all of a communication component 103 can be implemented to provide efficient functionality such as transmission functionality. For example, an antenna associated with the communication component 103 can be positioned at or near the outer side surface of the patch substrate 110 so as to allow transmission of signals with little or no absorption by the patch substrate 110.

FIGS. 11A-11C show examples of how the electroacoustic transducer 102 of FIG. 10 can be implemented utilizing one or more microphones. For the purpose of description of FIGS. 11A-11C, it will be understood that an electroacoustic transducer 102 can include one or more microphones 115 powered by a power source 116. In some embodiments, operation of such one or more microphones 115 can be facilitated by an assembly of one or more electronic circuits (114).

FIG. 11A shows that in some embodiments, a patch 100 can include a microphone 115 implemented to sense sound incident on the outer side of a patch substrate 110. Such a microphone can be at least partially exposed on the surface of the outer side, be covered with one or more protective layers, or some combination thereof. If covered with a protective layer, such a layer can be formed from material that provides a desirable transfer of the incident acoustic signal.

FIG. 11B shows that in some embodiments, a patch 100 can include a microphone 115 implemented to sense sound incident on the application side 111 of a patch substrate 110. Such a microphone can be at least partially exposed on the surface of the application side, be covered with one or more protective layers, or some combination thereof. If covered with a protective layer, such a layer can be formed from material that provides a desirable transfer of the incident acoustic signal.

FIG. 11C shows that in some embodiments, a patch 100 can include a microphone 115a implemented to sense sound incident on the outer side of a patch substrate 110, and a microphone 115b implemented to sense sound incident on the application side 111 of a patch substrate 110. The outer microphone 115a can be at least partially exposed on the surface of the outer side, be covered with one or more protective layers, or some combination thereof. The inner microphone 115b can be at least partially exposed on the surface of the application side 111, be covered with one or more protective layers, or some combination thereof. If covered with a protective layer, such a layer can be formed from material that provides a desirable transfer of the acoustic signal incident on the respective side of the patch substrate 110.

FIGS. 12A-12C depict examples of the patches of FIGS. 11A-11C sensing snoring sound(s) arriving through air, through body of a user, or combination thereof. For example, FIG. 12A shows a snoring sound sensing configuration that can be achieved with the patch 100 of FIG. 11A (applied to a skin portion 131 of a body 133 of a user). More particularly, snoring sound (indicated as 135) is depicted as arriving (e.g., through air) at a microphone 115 implemented at or near the outer side of a patch substrate 110.

In another example, FIG. 12B shows a snoring sound sensing configuration that can be achieved with the patch 100 of FIG. 11B (applied to a skin portion 131 of a body 133 of a user). More particularly, snoring sound (indicated as 137) is depicted as arriving through the body 133 (e.g., through tissue) at a microphone 115 implemented at or near the inner side of a patch substrate 110.

In yet another example, FIG. 12C shows a snoring sound sensing configuration that can be achieved with the patch 100 of FIG. 11C (applied to a skin portion 131 of a body 133 of a user). More particularly, snoring sound (indicated as 135) is depicted as arriving (e.g., through air) at a microphone 115a implemented at or near the outer side of a patch substrate 110, and snoring sound (indicated as 137) is depicted as arriving through the body 133 (e.g., through tissue) at a microphone 115b implemented at or near the inner side of a patch substrate 110.

It is noted that in some embodiments, sound signals arriving through the air and through the body, even if originating from the same user, may be distinguishable due to the difference in transmission mediums (e.g., between air and body tissue). Accordingly, in some embodiments, such a feature can be utilized to, for example, distinguish sources of snoring sounds among a plurality of users utilizing respective patches.

In some embodiments, a patch and/or a related system having one or more features as described herein can be configured to process a sensed sound to provide one or more desirable functionalities. In some embodiments, such processing of sensed sound can be achieved by the patch itself, by a related control unit associated with a separate device, or some combination thereof. As described herein, such processing of sensed sound can provide functionalities such as (1) identifying a snoring sound (compared to other sounds), (2) identifying a source of a snoring sound if there are more than one snoring sound sources, or some combination thereof.

For example, FIG. 13 shows that in some embodiments, a patch 100 can include a signal processing circuit 302 configured to process a signal obtained by a snoring sensor 102. For the purpose of description, such a snoring sensor can be configured as described herein (e.g., in reference to FIGS. 11 and 12). In some embodiments, the signal processing circuit 302 can be part of the electronics component 114 as described herein (e.g., in reference to FIGS. 11A-11C). Such an electronics component can further include a processor 300 configured to facilitate the operation of the signal processing circuit 302. In the example of FIG. 13, a communication component 103 can be provided to facilitate communication functionalities, including transmission of snoring activity information that has been at least partially processed by the signal processing circuit 302.

In another example, FIG. 14 shows that in some embodiments, a patch 100 can include a signal routing circuit 304 configured to route a signal obtained by a snoring sensor 102, to a control unit 152 associated with a separate device. For the purpose of description, such a snoring sensor can be configured as described herein (e.g., in reference to FIGS. 11 and 12). In some embodiments, the signal routing circuit 304 can be part of the electronics component 114 as described herein (e.g., in reference to FIGS. 11A-11C). Such an electronics component can further include a processor 300 configured to facilitate the operation of the signal routing circuit 304. In the example of FIG. 14, a communication component 103 can be provided to facilitate communication functionalities, including transmission of snoring activity information that has been routed by the signal routing circuit 304.

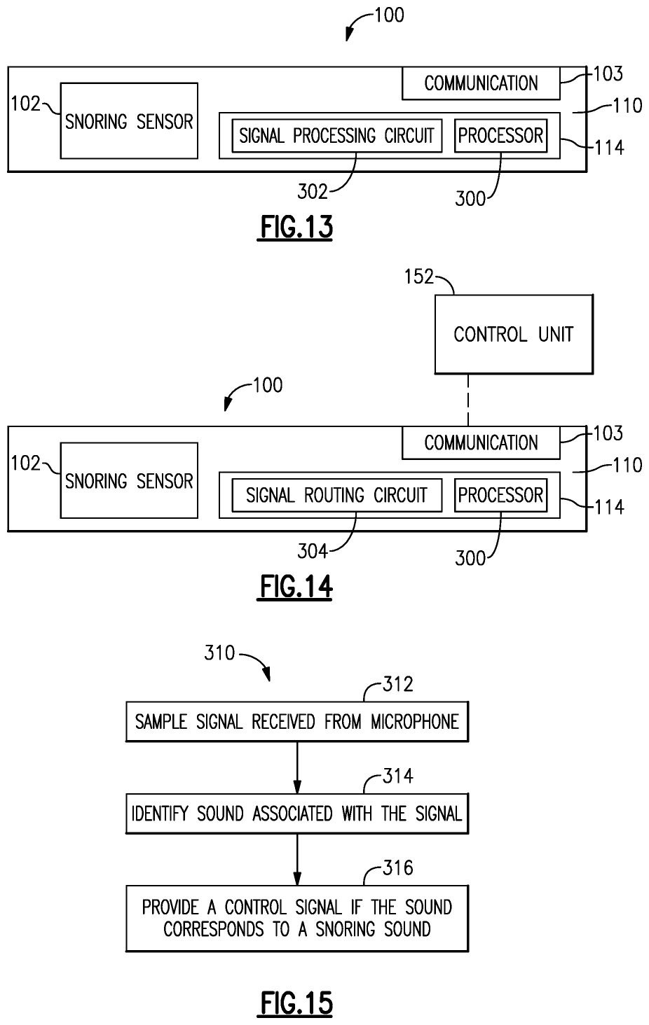

FIG. 15 shows a process 310 that can be implemented to identify a snoring sound, by a patch having one or more features as described herein. In some embodiments, such a process can be performed by the patch 100 and/or the related system described in reference to FIGS. 13 and 14.

Referring to FIG. 15, a signal received from a microphone of a patch can be sampled in block 312. Based on such sampling, sound associated with the microphone signal can be identified in block 314. If such an identified sound corresponds to a snoring sound, a control signal can be provided in block 316. As described herein, such a control signal can be utilized in a number of ways, including control of a snore-reduction action, monitoring of sleep/snoring activity, etc.

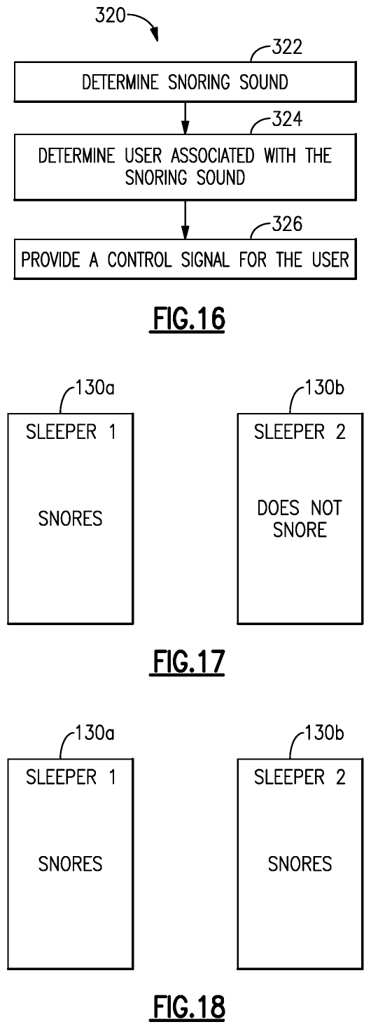

FIG. 16 shows a process 320 that can be implemented to identify a user associated with snoring sound, by a patch having one or more features as described herein. In some embodiments, such a process can be performed by the patch 100 and/or the related system described in reference to FIGS. 13 and 14.

Referring to FIG. 16, a snoring sound can be determined in block 322. In some embodiments, such a snoring sound can be determined with a process similar to the example of FIG. 15. Based on such a snoring sound determination, a user associated with the snoring sound can be determined in block 324. Based on such a user determination, a control signal can be provided for the user in block 326. As described herein, such a control signal can be utilized in a number of ways, including control of a snore-reduction action, monitoring of sleep/snoring activity, etc.

As described herein, a patch can include a snore sound sensing functionality, and such a feature can be utilized to benefit the user himself/herself, benefit another person nearby, or some combination thereof. In the context of two persons sleeping in close proximity, one person can be a snoring sleeper and the other person can be a non-snoring person, as depicted in FIG. 17; or both persons can be snoring sleepers, as depicted in FIG. 18. More particularly, in FIG. 17, a first person 130a can be a snoring person, and a second person 130b can be a non-snoring sleeper. In FIG. 18, both of first and second persons 130a, 130b can be snoring sleepers.

FIG. 19 shows an example of how a patch having one or more features as described herein can be utilized for the sleeping arrangement of FIG. 17. More particularly, a patch 100 as described herein can be worn by a snoring sleeper 130a, and the other person 130b who does not snore does not wear a patch. In the example of FIG. 19, the patch 100 is depicted as being configured similar to the example patch of FIG. 14, such that its snore sound sensing functionality is facilitated by an external control unit 152 in communication (151) with the patch 100.

Configured in the foregoing manner, a process 330 of FIG. 20 can be implemented. In block 332, snoring sound can be determined similar to the example process of FIG. 15. In block 334, any snoring sound determined in block 332 can be assigned to the snoring sleeper (130a in FIG. 19). In block 336, a control signal can be provided for the snoring sleeper to reduce or eliminate the snoring activity.

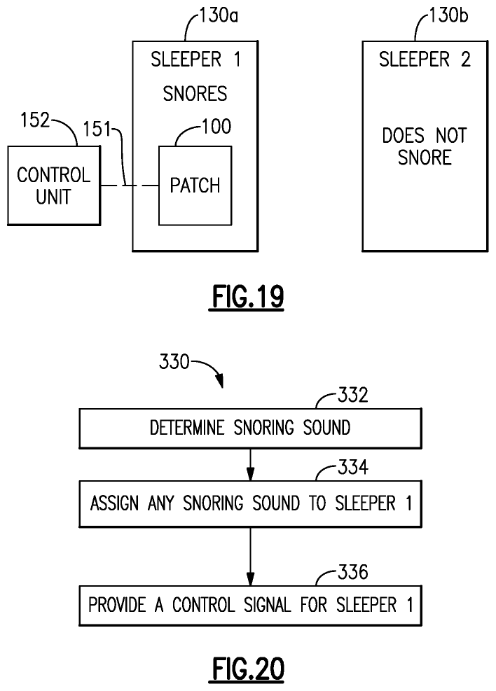

FIG. 21 shows an example of how a patch having one or more features as described herein can be utilized for the sleeping arrangement of FIG. 18. More particularly, a patch 100 as described herein can be worn by a first snoring sleeper 130a, and the other person 130b who also snores does not wear a patch. In the example of FIG. 21, the patch 100 is depicted as being configured similar to the example patch of FIG. 14, such that its snore sound sensing functionality is facilitated by an external control unit 152 in communication (151) with the patch 100.

In the foregoing example, a snoring sound detected by the patch 100 may be due to the patch-wearing and snoring sleeper 130a or the other snoring sleeper 130b. Accordingly, a process 340 of FIG. 22 can be implemented. In block 342, snoring sound can be determined similar to the example process of FIG. 15. In block 344, the process 340 can determine whether the detected snoring sound is from the patch-wearing sleeper or the other snoring sleeper. In block 346, a control signal can be provided for the patch-wearing sleeper to reduce or eliminate the snoring activity of the patch-wearing sleeper.

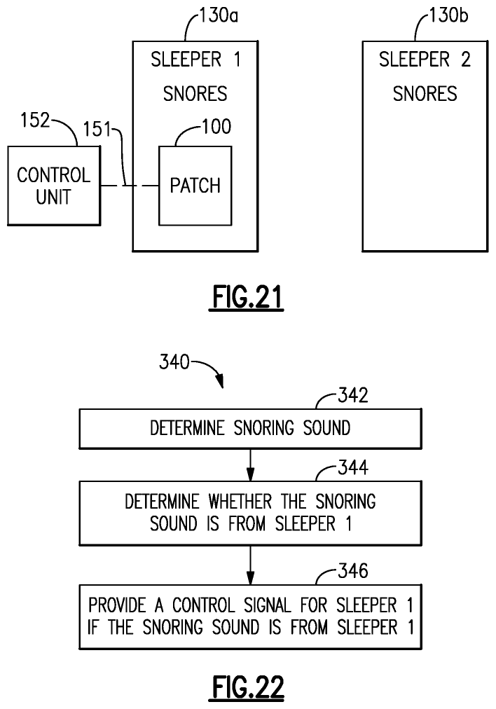

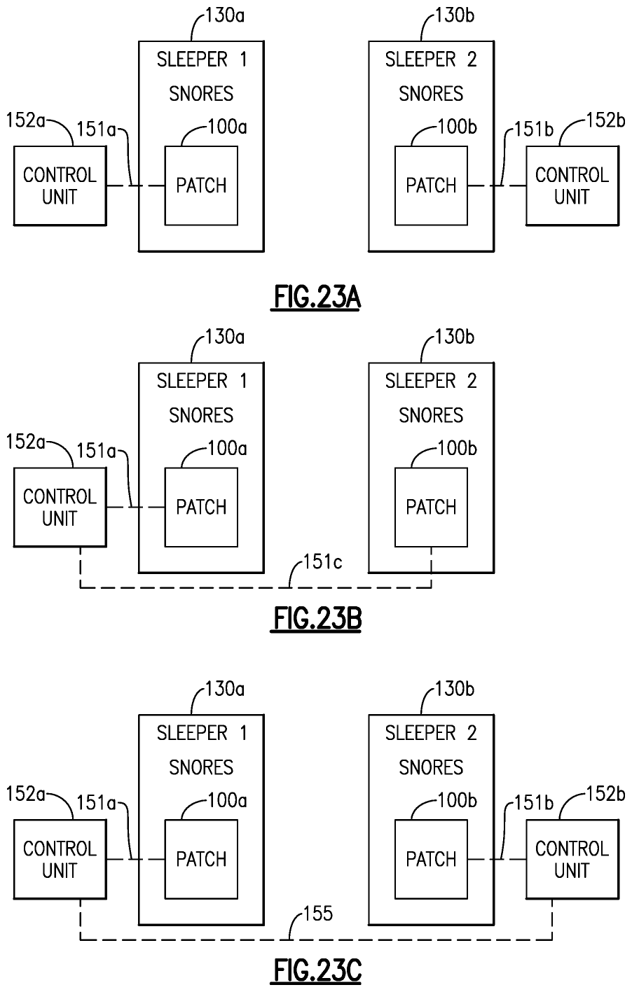

FIGS. 23A-23C show an example sleeping arrangement that is similar to the example of FIG. 21, but in which each of the two snoring sleepers 130a, 130b wears a respective patch 100a, 100b. FIGS. 23A-23C also show various non-limiting examples of how such patches worn by two sleepers can communicate with one or more control units.

For example, FIG. 23A shows a configuration where each patch has a dedicated control unit. Accordingly, the first patch 100a worn by the first sleeper 130a can communicate (151a) with a first control unit 152a, and the second patch 100b worn by the second sleeper 130b can communicate (151b) with a second control unit 152b. In such an example configuration, each patch/control unit pair can be configured to operate in a manner similar to the example of FIGS. 21 and 22.

In another example, FIG. 23B shows a configuration where a common control unit 152a communicates with both of the patches 100a, 100b worn by the first and second sleepers 130a, 130b. More particularly, the control unit 152a is shown to communicate (151a) with the first patch 100a worn by the first sleeper 130a, and also communicate (151c) with the second patch 100b worn by the second sleeper 130b. In such a configuration, each patch can be configured to provide an identifying information to allow the common control unit 152a to process signals from both patches.

In yet another example, FIG. 23C shows a configuration that is similar to the example of FIG. 23A, but in which the two control units 152a, 152b can communicate (155) with each other. In such a configuration, each patch can be configured to provide an identifying information to allow the control units 152a, 152b to share information associated with the two patches.

It will be understood that other combinations of communication links can be established among a plurality of patches and one or more control units.

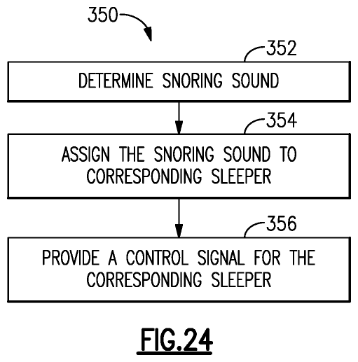

Configured in the foregoing manner, a process 350 of FIG. 24 can be implemented. In block 352, snoring sound can be determined for a given patch similar to the example process of FIG. 15. In block 354, the snoring sound determined in block 352 can be assigned to a corresponding sleeper (130a or 130b in FIGS. 23A-23C). In block 356, a control signal can be provided for the respective snoring sleeper to reduce or eliminate the snoring activity.

As described herein, a patch having a snoring sound sensor can be utilized to determine whether a detected sound is a snoring sound or not a snoring sound. In some embodiments, such sound identifying determination can be achieved in a number of ways, including, for example, sound pattern (e.g., in time and/or frequency domain), sound intensity level, presence/absence of vibrations/sound waves in tissue covered by the patch, etc.

As also described herein, a patch having a snoring sound sensor can be utilized to distinguish one snoring sound from another snoring sound. In some embodiments, such snoring sound source identifying determination can be achieved based on detected snoring sound, with information associated with a user wearing the patch, or some combination thereof. For example, suppose there are two snoring sleepers generating respective snoring sounds. A patch associated with one sleeper may detect both snoring sounds that are sufficiently distinguishable based on, for example, sound patterns (e.g., in time and/or frequency domain), sound intensity levels, presence/absence of vibrations/sound waves in tissue covered by the patch, etc. In such an example, it is noted that the snoring sound source determination can be achieved without use of prior information about the sleeper wearing the patch.

FIGS. 25-27 show examples where a personalized profile can be implemented for a user wearing a patch having one or more features as described herein. In some embodiments, such a personalized profile can include information unique to the user so as to facilitate identification of the user as a source of snoring sound. Such unique information can be based on some or all of the various snoring sound identifying properties as described herein.

FIG. 25 shows an example configuration where a patch 100 is associated with a personalized profile 360 implemented in or accessible by a control unit 152. In such a configuration, the control unit 152 can communicate (151) with the patch 100 to build and/or updated the personalized profile 360 of a user, utilize the personalized profile 360 to identify (or reject) the user as a source of a snoring sound, or some combination thereof.

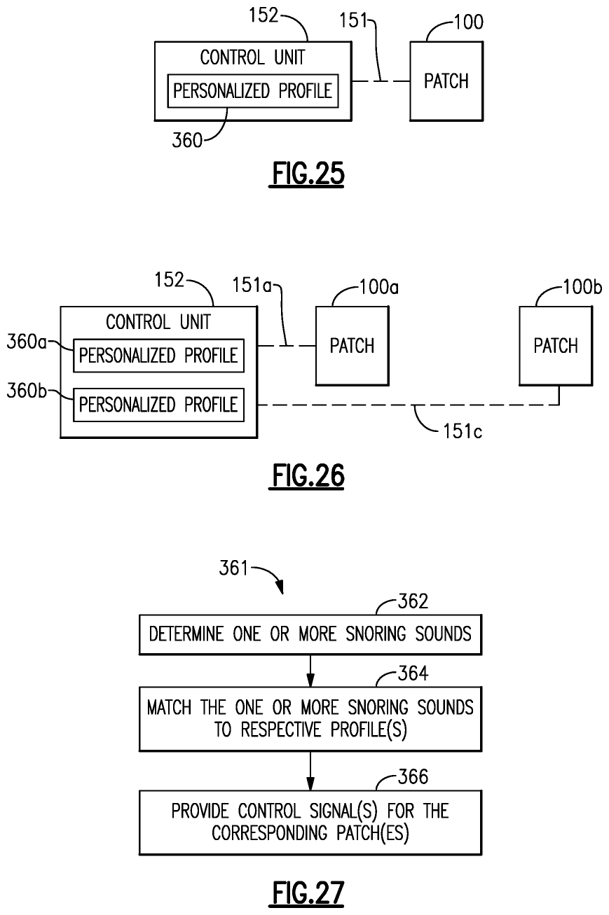

FIG. 26 shows that in some embodiments, a control unit 152 can include or have access to a plurality of personalized profiles associated with respective patches for use with different users. For example, the control unit 152 is shown to include a first personalized profile 360a associated with a first patch 100a, and such an association is depicted by a first communication link 151a. Similarly, the control unit 152 is shown to also include a second personalized profile 360b associated with a second patch 100b, and such an association is depicted by a second communication link 151c. It will be understood that the first and second communication links 151a, 151c can be achieved by separate channels, through a common channel with patch identification information, or some combination thereof.

FIG. 27 shows a process 361 that can be implemented for the example configurations of FIGS. 25 and 26. In block 362, one or more snoring sounds can be determined. In block 364, each of such snoring sounds determined in block 362 can be matched with a corresponding personalized profile. In block 366, the personalized profile can be updated based on the match of block 362 if such a personalized profile is being built, or a control signal can be provided for a user of the corresponding patch based on the match of block 362 if the personalized profile is being utilized for snoring sound source identification purpose.

In the examples described in reference to FIGS. 25-27, a personalized profile is depicted as being associated with a respective control unit. It will be understood that in some embodiments, some or all of such a personalized profile can be implemented in the patch itself.

In some embodiments, a personalized profile, whether implemented as part of a control unit separate from a patch or as part of the patch itself, can include information specific to a user using the patch. Such information can include some or all of, for example, a log of snoring activity over a selected period of time, nature of snoring activity (e.g., pattern, loudness, etc.), a log of actions taken in response to detected snoring activity, etc. Such information can be utilized to, for example, enhance effectiveness of stimulus being provided to reduce or eliminate snoring, enhance snoring detection efficiency, etc. In some applications, such information can be utilized independent of any stimulus generation. For example, snoring log can be reviewed by a professional to provide additional information related to a sleep study.

In some implementations, a patch having one or more features as described herein can be utilized to activate a stimulus for a snoring user wearing the patch. Such a stimulus can be, for example, a change in sleeping position, one or more sensory stimulus such as touch and/or sound, and/or any stimulus that can reduce reduction or elimination of the snoring activity of the user. Non-limiting examples of activations of snoring-reducing stimuli are described herein in greater detail.

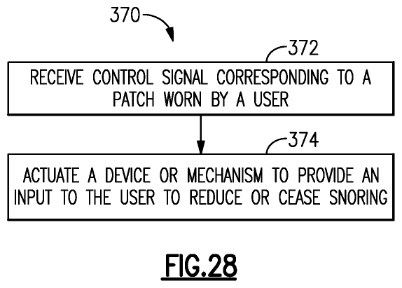

FIG. 28 shows a process 370 that can be implemented to actuate a device or mechanism to provide a stimulus to a sleeping user wearing a patch. For the purpose of description, it will be understood that such a patch has sensed a snoring activity of the user, and information related to such a sensed snoring activity has been provided to a controller.

In block 372, a control signal corresponding to a patch (worn by a user) can be received by a device or mechanism. In block 374, the device or mechanism can be actuated to provide an input to the user to reduce or cease the snoring activity.

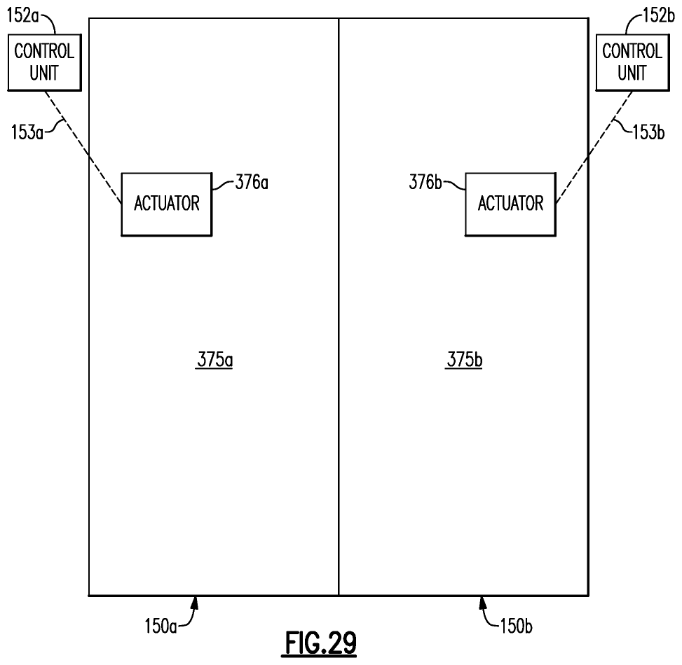

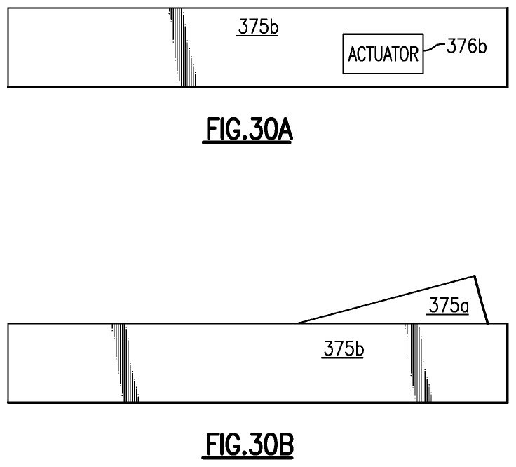

FIGS. 29 and 30 show an example where an activation of snoring-reducing stimulus can be provided by a bed. In some embodiments, a bed can include a first sleeping portion 150a and a second sleeping portion 150b for first and second sleepers, respectively. Each of such sleeping portions can be controlled independently so as to provide a set of one or more sleeping parameters such as sleeping orientation and mattress firmness.

FIG. 29 depicts a block diagram of a bed having the foregoing first and second sleeping portions 150a, 150b. The first sleeping portion 150a can include a first mattress 375a, and a first actuator 375a configured to effectuate one or more adjustments for the first mattress 375a and thus for the first sleeper sleeping thereon. Similarly, the second sleeping portion 150b can include a second mattress 375b, and a second actuator 375b configured to effectuate one or more adjustments for the second mattress 375b and thus for the second sleeper sleeping thereon.

As depicted in FIG. 29, the first actuator 376a can be in communication (153a) with a first control unit 152a, and such a first control unit can be in communication with a first patch worn by the first sleeper. Similarly, the second actuator 376b can be in communication (153b) with a second control unit 152b, and such a second control unit can be in communication with a second patch worn by the second sleeper. Accordingly, snoring activity of one sleeper can be detected by the respective patch, and a resulting control signal generated by the corresponding control unit can be provided to the respective actuator to thereby effectuate a change in one or more sleeping parameters for the snoring sleeper. It will be understood that such a change in one or more sleeping parameters can be effectuated without any involvement of the other sleeper, thereby enhancing the quality of sleep for both of the sleepers.

For example, suppose that the first sleeper (on the first mattress 375a) snores, and the second sleeper (on the second mattress 375b) does not snore. Also suppose that initially, both sleepers have their respective mattresses in a generally flat configuration, as depicted in the side view of FIG. 30A. When the first sleeper's snoring activity is detected by a patch worn by that sleeper, the resulting control signal provided by the control unit (152a in FIG. 29) to the corresponding actuator 376a can result in the first mattress 375a to be adjusted to reduce or eliminate further snoring of the first sleeper. For example, the first mattress 375a can be adjusted, as depicted in FIG. 30B, so as to raise the upper portion of the first sleeper.

In the example of FIGS. 29 and 30, it is assumed that a patch worn by the snoring sleeper and the corresponding control unit effectuate the change in the corresponding mattress configuration to reduce or eliminate the snoring activity of the same sleeper. However, it will be understood that other patch/control unit/actuator arrangements can also be utilized. For example, suppose that the snoring sleeper is not able to, or does not wish to, wear a patch. In such a situation, a patch having one or more features as described herein can be worn by the other sleeper, even though that other sleeper does not snore. In such a configuration, the patch worn by the other sleeper can be utilized to control the actuator associated with the mattress of the snoring sleeper. In some embodiments, such a control functionality can be achieved through a control unit associated with the other sleeper (non-snorer) (e.g., such a control unit can communicate with the actuator associated with the snoring sleeper), a control unit associated with the snoring sleeper (e.g., the patch worn by the non-snorer can communicate with the control unit associated with the snoring sleeper), or some combination thereof.

In some embodiments of the example of FIGS. 29 and 30, at least the actuators 376a, 376b can be integrated as parts of the bed. In some embodiments, some or all of the control functionalities associated with the control units 152a, 152b may be integrated as parts of the bed, may be implemented in one or more separate devices, or some combination thereof.

FIG. 31 shows an example where an activation of snoring-reducing stimulus can be provided by a device that may or may not be an integral part of a bed. In some embodiments, such a bed can include a first sleeping portion 150a and a second sleeping portion 150b for first and second sleepers, respectively. Each of such sleeping portions (150a, 150b) can include a pad having a plurality of transducers 382 configured to, for example, sense sleeping position, provide mechanical stimulus for the sleeper, or some combination thereof. In some embodiments, such a pad can be an integral part of the bed, a separate device that can be implemented on top of an existing mattress, or some combination thereof.

In the example of FIG. 31, the first pad 380a can be in communication (153a) with a first control unit 152a, and such a first control unit can be in communication with a first patch worn by the first sleeper. Similarly, the second pad 380b can be in communication (153b) with a second control unit 152b, and such a second control unit can be in communication with a second patch worn by the second sleeper. Accordingly, snoring activity of one sleeper can be detected by the respective patch, and a resulting control signal generated by the corresponding control unit can be provided to the respective pad to thereby provide a stimulus for the snoring sleeper. It will be understood that such a stimulus can be effectuated without any involvement of the other sleeper, thereby enhancing the quality of sleep for both of the sleepers.

For example, suppose that the first sleeper (on the first pad 380a) snores, and the second sleeper (on the second pad 380b) does not snore. When the first sleeper's snoring activity is detected by a patch worn by that sleeper, the resulting control signal provided by the control unit 152a to the corresponding pad 380a can result in the first pad 380a providing a stimulus to the first sleeper to reduce or eliminate further snoring of the first sleeper.

In some embodiments, the stimulus provided by the pad 380a to the snoring sleeper can be configured to reduce or eliminate further snoring in a direct manner. For example, mechanical stimulus such as vibration of one or more transducers 382 provided to the snoring sleeper can directly cause the sleeper to cease or reduce the sleeping activity.

In some embodiments, the stimulus provided by the pad 380a to the snoring sleeper can be configured to reduce or eliminate further snoring in an indirect manner. For example, one or more transducers 382 can be utilized to sense the orientation of the snoring sleeper's body, and/or induce the snoring sleeper to change the orientation of the snoring sleeper's body to a new orientation where snoring activity is reduced or eliminated. For example, suppose that the sleeper snores while sleeping on the back. Upon detection of the snoring activity, a control signal provided to the pad 380a can cause one or more transducers to provide a stimulus to the sleeper to induce the sleeper to turn the body (e.g., sleeping on the back to sleeping on the side) to reduce or eliminate further snoring. In the context of the example where the sleeper is induced into turning the body, in some embodiments, the pad 380a can be actuated to cause such a turning so that the body turns so as to have the snoring sleeper face away from the other (non-snoring) sleeper.

In the example of FIG. 31, it is assumed that a patch worn by the snoring sleeper and the corresponding control unit effectuate the operation of the corresponding pad to reduce or eliminate the snoring activity of the same sleeper. However, it will be understood that other patch/control unit/pad arrangements can also be utilized. For example, suppose that the snoring sleeper is not able to, or does not wish to, wear a patch. In such a situation, a patch having one or more features as described herein can be worn by the other sleeper, even though that other sleeper does not snore. In such a configuration, the patch worn by the other sleeper can be utilized to control the pad associated with the snoring sleeper. In some embodiments, such a control functionality can be achieved through a control unit associated with the other sleeper (non-snorer) (e.g., such a control unit can communicate with the pad associated with the snoring sleeper), a control unit associated with the snoring sleeper (e.g., the patch worn by the non-snorer can communicate with the control unit associated with the snoring sleeper), or some combination thereof.

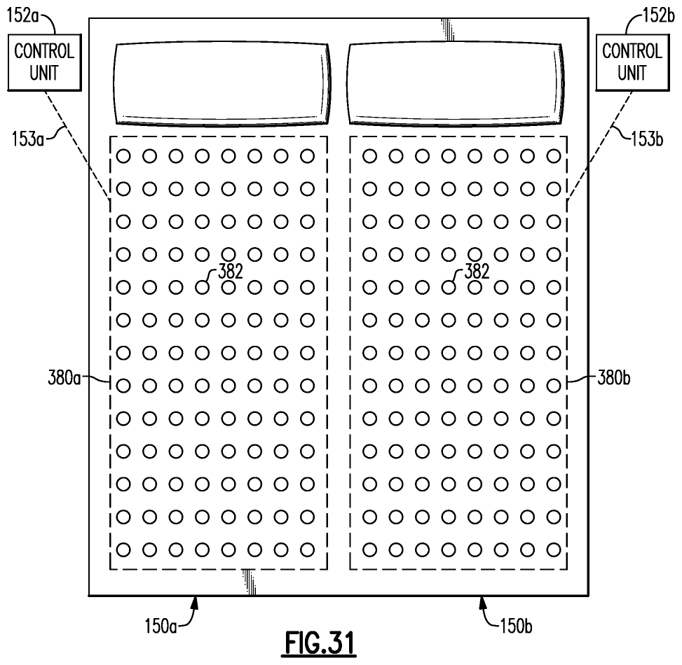

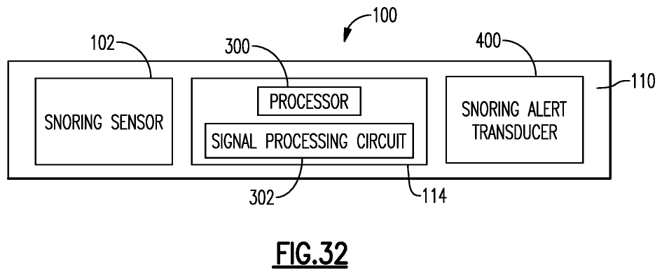

In the examples of FIGS. 29-31, a patch can be configured to sense a snoring activity as described herein, and based on such sensing, stimulus can be provided to a snoring person by a separate device. FIG. 32 shows that in some embodiments, a patch 100 having a snoring sensor 102 as described herein can also include a stimulus providing component 400. In some embodiments, such a stimulus providing component can include, for example, one or more transducers configured to provide an alert of the snoring activity and/or reduce or eliminate the snoring activity. In some embodiments, such a stimulus can include a low-intensity sound configured to alert the snoring sleeper (but not the other (non-snoring) sleeper), and/or a vibration that can be utilized to reduce or eliminate the snoring activity. Among others, examples of such alert functionalities are described in PCT Publication No. WO 2019/023360 titled WEARABLE PATCHES FOR SLEEP APPLICATIONS, which is expressly incorporated by reference in its entirely, and its disclosure is to be considered part of the specification of the present application.

In the example of FIG. 32, the patch 100 can include a patch substrate 110 as described herein, and electronics 114 having a processor 300 and a signal processing circuit 302, similar to the example of FIG. 13. In FIG. 32, however, since the patch 100 includes the stimulus providing component 400, the patch 100 may or may not include a communication component for communicating with an external control unit.

FIG. 33 shows that in some embodiments, one or more patches having one or more features as described herein can be in a packaged format 502 for easier use by a user. Such a packaged format of patch(es) can be included in, for example, a packaged product 500. In some embodiments, the packaged product 500 can also include an instruction 504 such as a printed instruction. Such an instruction can provide information on, for example, proper and/or recommended application of the included patch(es).

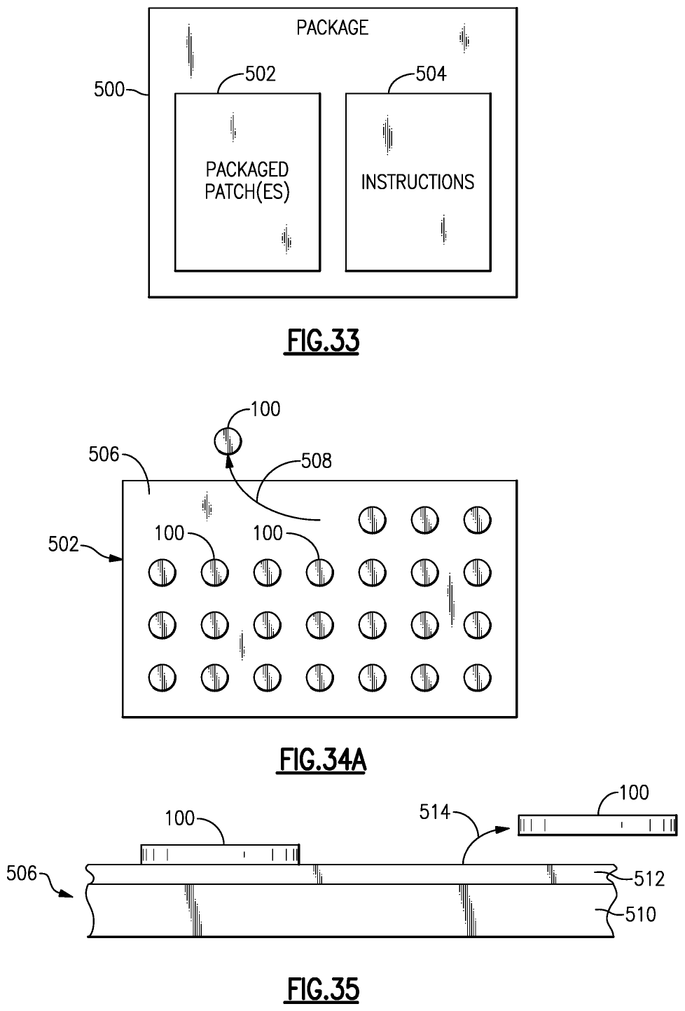

FIG. 34A shows an example of a packaged format 502 having a support sheet 506 with a plurality of patches 100 secured thereto. Such number of patches can allow a user to remove (arrow 508) a patch 100 from the support sheet 506 for use whenever snoring reduction or elimination is desired. In some applications, such use of patches can be performed for an unspecified number of days, only as needed or desired, for a specified number of days to build a snoring profile of the user, or any combination thereof.

FIG. 34B shows that in some embodiments, each patch 100 in the example of FIG. 34A can include an adhesive element 100a and one or more active elements 100b configured to provide one or more functionalities as described herein. In some embodiments, such one or more active elements can include electronic and mechanical components configured to, for example sense snoring of a user when the patch is attached to the user with the adhesive element 100a. In some embodiments, the adhesive element 100a can also allow the patch 100 to be secured to a support sheet (e.g., 506 in FIG. 34A) until it is removed for use.

FIG. 34C shows a packaged product 500 that can be a more specific example of the packaged product 500 of FIG. 33. Such a packaged product can include a plurality of support sheets 506 with each support sheet having a plurality of patches 100 secured thereto to provide an example packaged format. Each patch 100 can be configured to include one or more features as described herein.

FIG. 35 shows an enlarged side sectional view of an example support sheet 506 that can be utilized to hold (until removal) a plurality of patches, similar to the example of FIG. 34A. In some embodiments, the support sheet 506 can include a base layer 510 (e.g., paper, plastic, etc.) and a release layer 512. The release layer 512 can be secured to the base layer 510, and be configured to securely hold the patches 100 thereon during transport and storage phases. Assuming that a patch includes an adhesive layer for application onto the skin of a user, the release layer can further be configured to allow the patch to be removed (e.g., peeled off) cleanly for application onto the user. In the example of FIG. 35, such removal of the patch 100 from the release layer 512 is depicted as an arrow 514.

In some embodiments, it may be desirable to activate a patch at an appropriate time (e.g., when removed from a release layer or when applied to the skin of a user). For example, such an activation can include a hand-shake pairing process between the patch and a control unit. In some embodiments, such a hand-shake paring process can be initiated when a patch is removed from a release layer, and when the patch is in appropriate proximity to a control unit.

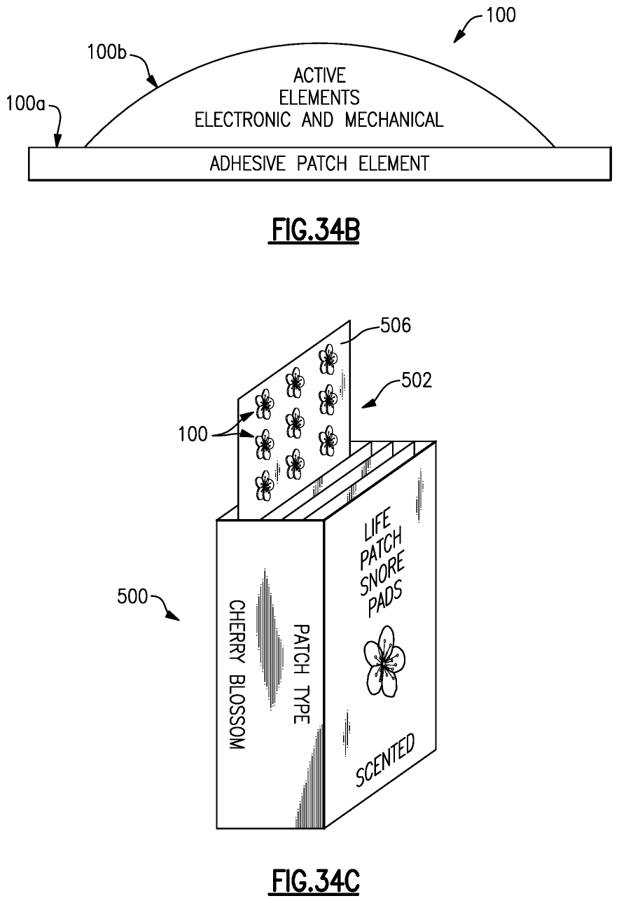

FIG. 36 shows a process 520 that can be implemented in a patch. In block 522, an activation condition can be detected. In block 524, a pairing process can be initiated.

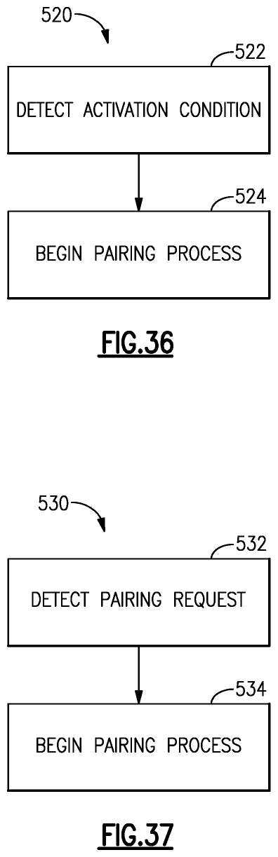

FIG. 37 shows a process 530 that can be implemented in a controller. In block 532, a pairing request can be detected (e.g., from a patch, as a result of block 524 of the process 520). In block 534, a pairing process can be carried out with the patch.

In the examples of FIGS. 36 and 37, the patch can be configured to be able to detect an activation condition by itself. However, in some embodiments, a patch can include an RFID circuitry that remains unpowered until interrogated by an external device such as a control unit. For such a configuration of the patch, some or all of the process 520 of FIG. 36 can be triggered and performed upon an interrogation by the control unit.

FIG. 38 depicts a block diagram of a system that can include a non-invasive patch 100 having one or more features as described herein. In some embodiments, such a system can also include an external control unit 152 configured to communicate with the non-invasive patch 100 to, for example, provide an snoring-reduction control signal.

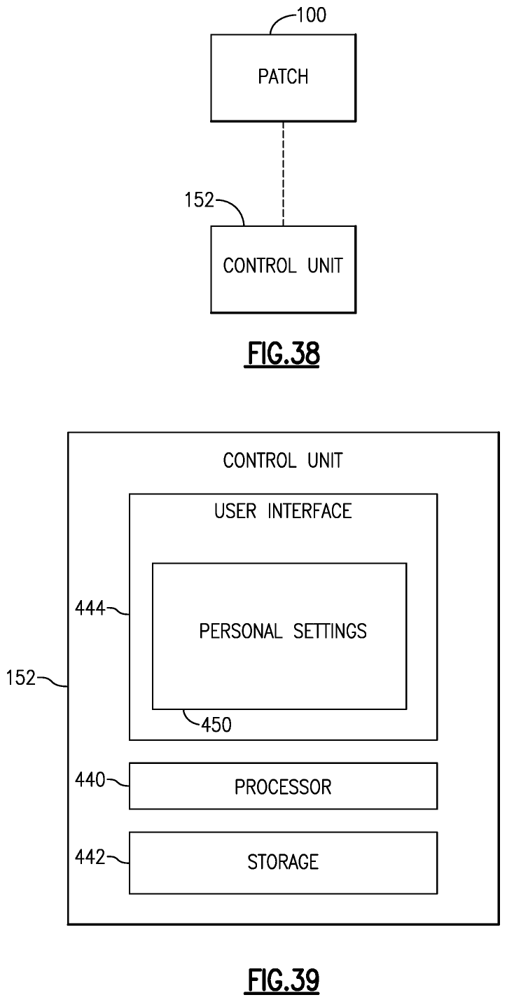

FIG. 39 depicts a control unit 152 that can be utilized as the control unit 152 of the system of FIG. 38. In some embodiments, the control unit 152 of FIG. 39 can include a user interface 444 configured to allow a user to control one or more functionalities associated with a corresponding patch (e.g., 100 in FIG. 38). Such functionalities can include, for example, turning activation control ON or OFF, setting of stimulus properties such as type, intensity, duration, etc. In the example of FIG. 39, some or all of such stimulation functionalities can be facilitated by a personal settings component 450 of the user interface 444.

As further shown in the example of FIG. 39, the control unit 152 can include a processor 440; and such a processor can provide and/or facilitate some or all of the foregoing control functionalities. The control unit 152 can further include a memory or storage component 442 (e.g., a non-transitory computer readable medium); and such a storage component can store information such as stimulus settings.

In some embodiments, the control unit 152 of FIG. 39 can be implemented as a dedicated device configured for operation with one or more patches. Such a dedicated device can generate stimulus control signals based on the corresponding patch(es).

In some embodiments, the control unit 152 of FIG. 39 can be implemented as an application software (also referred to as an app) running on a device such as a smartphone. Similar to the dedicated device described above, the application software can be configured to generate stimulus control signals based on sensing achieved by one or more patches.

As described herein, a patch having one or more features as described herein can include a communication component to facilitate transmission of information such as sensor data, and/or to facilitate reception of information such as patch settings. FIG. 40 shows an example of a system that can be implemented to utilize such a communication functionality. For example, a patch 100 having one or more features as described herein is shown to be worn by a user 830. Information transmitted (e.g., in a wireless manner) is depicted as 880, and such information can be received by a monitor 152 (also referred to herein as a control unit). Such a monitor can include a receiver circuit configured to process the received signal from the patch 100. The monitor 152 can further include a processor to support various functionalities as described herein.

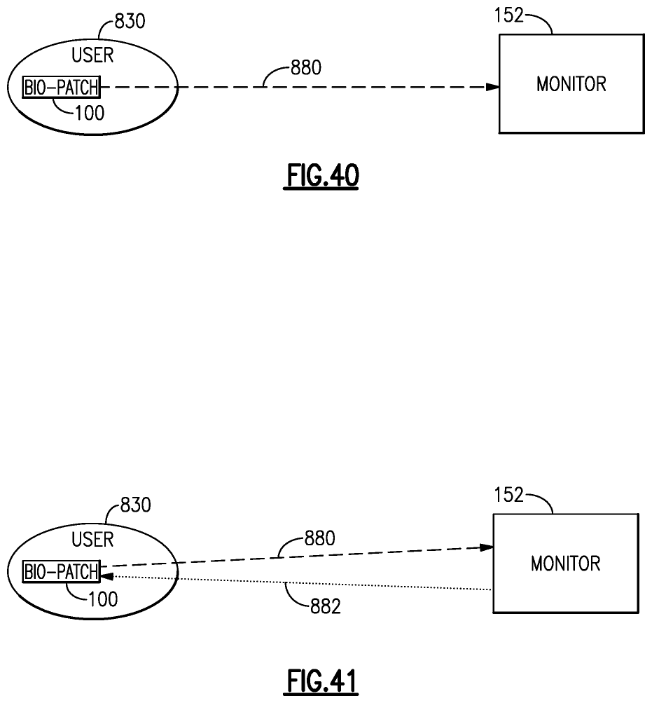

In some embodiments, a patch having one or more features as described herein can also include a receiver circuit to allow the patch to receive information such as instructions, diagnostics, etc. Accordingly, FIGS. 41 shows an example of a system that can be implemented to utilize such transmit and receive functionalities. For example, a patch 100 having one or more features as described herein is shown to be worn by a user 830. Information transmitted (e.g., in a wireless manner) is depicted as 880, and such information can be received and processed by a monitor 152, similar to the example of FIG. 40.

In the example of FIG. 41, the patch 100 can also receive information (indicated as 882). Such received information can be achieved in a wireless mode, a wire mode, or any combination thereof. Although such information is depicted as being provided by the monitor 152, it will be understood that information provided to the patch 100 may or may not be from the same component (e.g., monitor in FIG. 41).

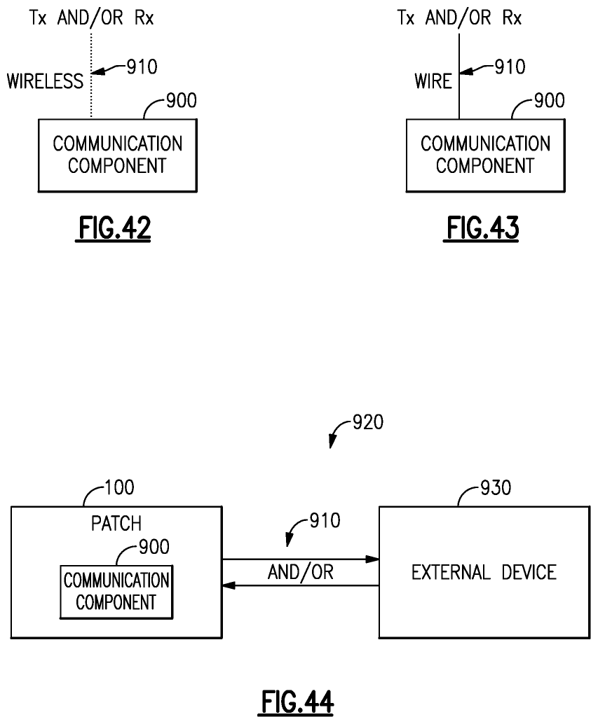

FIGS. 42-45 show examples of communications and/or system functionalities that can be implemented in a system having one or more patches as described herein. For example, FIGS. 42 and 43 show that in some embodiments, a communication component 900 of a patch can be configured to provide a wireless communication (depicted as 910 in FIG. 42) with an external device, a wired communication (depicted as 910 in FIG. 43) with an external device, or some combination thereof. For the purpose of description of FIGS. 42 and 43, an external device can be another patch, a non-patch device, etc.

In some embodiments, in each of the examples of FIGS. 42 and 43, the wireless and/or wired communication link 910 can include a transmit (Tx) functionality (relative to the corresponding patch), a receive (Rx) functionality, or any combination thereof.

FIG. 44 shows a system 920 that can be formed with one or more patches 100 as described herein, and an external device 930. For the purpose of description of FIG. 44, it will be understood that the external device 930 is relative to the patch 100. Thus, if the external device 930 is another patch, then the patch 100 shown in FIG. 44 can be considered to be external to the other patch (930). As described in reference to FIGS. 42 and 43, it will be understood that the external device 930 can be a patch that may or may not be similar to the patch 100.

In the example of FIG. 44, the patch 100 is shown to include a communication component similar to the examples of FIGS. 42 and 43. Accordingly, the communication between the patch 100 and the external device 930 can include transmit and/or receive portions.

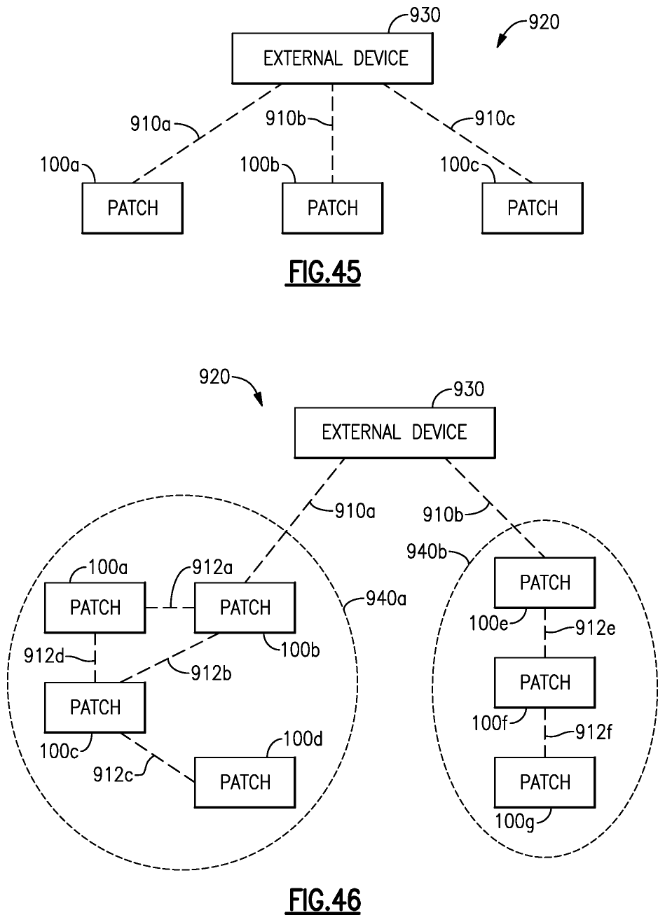

FIG. 45 shows that in some embodiments, the system 920 of FIG. 44 can include a plurality of patches that communicate with a common external device. For example, a system 920 of FIG. 45 is shown to include a plurality of patches 100a, 100b, 100c and an external device 930. More particularly, the first patch 100a can be in communication (910a) with the external device 930, the second patch 100b can be in communication (910b) with the external device 930, and the third patch 100c can be in communication (910c) with the external device 930. In some embodiments, such an external device can be configured to, for example, coordinate operations of the patches (100a, 100b, 100c), collect data from the patches, etc. In some embodiments, the external device 930 can be configured to communicate with another device at a similar level, with another device at a higher level, or any combination thereof.

FIG. 46 shows that in some embodiments, the system 920 of FIG. 44 can include a plurality of patches that can communicate with each other, and/or with an external device. For example, a first group (940a) of patches and a second group (940b) are shown to be included in a system 920, and generally in communication with an external device 930. More particularly, the first group 940a is shown to include four example patches 100a, 100b, 100c, 100d, and the second group 940b is shown to include three example patches 100e, 100f, 100g. Such first and second groups 940a, 940b of patches can be grouped based on, for example, physical proximity/separation, different functionalities, etc.

In some embodiments, within a given group, each of the plurality of patches can communicate directly with the external device 930, through a representative patch, or some combination thereon. For example, for the first group 940a, the patches 100a and 100b are shown to have a communication link 912a; the patches 100a and 100c are shown to have a communication link 912d; the patches 100c and 100d are shown to have a communication link 912c; and the patches 100c and 100b are shown to have a communication link 912b. Further, the patch 100b is shown to be a representative communication member and be in communication (910a) with the external device 930.

In another example, for the second group 940b, the patches 100e and 100f are shown to have a communication link 912e; and the patches 100f and 100g are shown to have a communication link 912f. Further, the patch 100e is shown to be a representative communication member and be in communication (910b) with the external device 930.

In some embodiments, the communication links between the patches within a given group can be based on, for example, different patches worn by a given user, relative proximity/distance among the users wearing the respective patches, some hierarchy of the users and/or patches, or some combination thereof. In some embodiments, the communication links between the patches can be configured as a mesh network, or be based on such a network.

In some embodiments, a system of patches as described herein (e.g., in reference to FIGS. 42-46) can provide a system level information that may not be available from an individual patch.

The present disclosure describes various features, no single one of which is solely responsible for the benefits described herein. It will be understood that various features described herein may be combined, modified, or omitted, as would be apparent to one of ordinary skill. Other combinations and sub-combinations than those specifically described herein will be apparent to one of ordinary skill, and are intended to form a part of this disclosure. Various methods are described herein in connection with various flowchart steps and/or phases. It will be understood that in many cases, certain steps and/or phases may be combined together such that multiple steps and/or phases shown in the flowcharts can be performed as a single step and/or phase. Also, certain steps and/or phases can be broken into additional sub-components to be performed separately. In some instances, the order of the steps and/or phases can be rearranged and certain steps and/or phases may be omitted entirely. Also, the methods described herein are to be understood to be open-ended, such that additional steps and/or phases to those shown and described herein can also be performed.

Some aspects of the systems and methods described herein can advantageously be implemented using, for example, computer software, hardware, firmware, or any combination of computer software, hardware, and firmware. Computer software can comprise computer executable code stored in a computer readable medium (e.g., non-transitory computer readable medium) that, when executed, performs the functions described herein. In some embodiments, computer-executable code is executed by one or more general purpose computer processors. A skilled artisan will appreciate, in light of this disclosure, that any feature or function that can be implemented using software to be executed on a general purpose computer can also be implemented using a different combination of hardware, software, or firmware. For example, such a module can be implemented completely in hardware using a combination of integrated circuits. Alternatively or additionally, such a feature or function can be implemented completely or partially using specialized computers designed to perform the particular functions described herein rather than by general purpose computers.

Multiple distributed computing devices can be substituted for any one computing device described herein. In such distributed embodiments, the functions of the one computing device are distributed (e.g., over a network) such that some functions are performed on each of the distributed computing devices.

Some embodiments may be described with reference to equations, algorithms, and/or flowchart illustrations. These methods may be implemented using computer program instructions executable on one or more computers. These methods may also be implemented as computer program products either separately, or as a component of an apparatus or system. In this regard, each equation, algorithm, block, or step of a flowchart, and combinations thereof, may be implemented by hardware, firmware, and/or software including one or more computer program instructions embodied in computer-readable program code logic. As will be appreciated, any such computer program instructions may be loaded onto one or more computers, including without limitation a general purpose computer or special purpose computer, or other programmable processing apparatus to produce a machine, such that the computer program instructions which execute on the computer(s) or other programmable processing device(s) implement the functions specified in the equations, algorithms, and/or flowcharts. It will also be understood that each equation, algorithm, and/or block in flowchart illustrations, and combinations thereof, may be implemented by special purpose hardware-based computer systems which perform the specified functions or steps, or combinations of special purpose hardware and computer-readable program code logic means.

Furthermore, computer program instructions, such as embodied in computer-readable program code logic, may also be stored in a computer readable memory (e.g., a non-transitory computer readable medium) that can direct one or more computers or other programmable processing devices to function in a particular manner, such that the instructions stored in the computer-readable memory implement the function(s) specified in the block(s) of the flowchart(s). The computer program instructions may also be loaded onto one or more computers or other programmable computing devices to cause a series of operational steps to be performed on the one or more computers or other programmable computing devices to produce a computer-implemented process such that the instructions which execute on the computer or other programmable processing apparatus provide steps for implementing the functions specified in the equation(s), algorithm(s), and/or block(s) of the flowchart(s).

Some or all of the methods and tasks described herein may be performed and fully automated by a computer system. The computer system may, in some cases, include multiple distinct computers or computing devices (e.g., physical servers, workstations, storage arrays, etc.) that communicate and interoperate over a network to perform the described functions. Each such computing device typically includes a processor (or multiple processors) that executes program instructions or modules stored in a memory or other non-transitory computer-readable storage medium or device. The various functions disclosed herein may be embodied in such program instructions, although some or all of the disclosed functions may alternatively be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list. The word “exemplary” is used exclusively herein to mean “serving as an example, instance, or illustration.” Any implementation described herein as “exemplary” is not necessarily to be construed as preferred or advantageous over other implementations.

The disclosure is not intended to be limited to the implementations shown herein. Various modifications to the implementations described in this disclosure may be readily apparent to those skilled in the art, and the generic principles defined herein may be applied to other implementations without departing from the spirit or scope of this disclosure. The teachings of the invention provided herein can be applied to other methods and systems, and are not limited to the methods and systems described above, and elements and acts of the various embodiments described above can be combined to provide further embodiments. Accordingly, the novel methods and systems described herein may be embodied in a variety of other forms; furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure.

1. A system for reducing snoring activity, the system comprising:
a patch configured to be secured to a skin of a user and generate a first signal indicative of a snoring sound; and
a control unit configured to generate a second signal based on the first signal, the second signal configured to allow actuation of a stimulus that reduces snoring activity associated with the snoring sound.

2. (canceled)

3. (canceled)

4. The system of claim 1 wherein the control unit includes a device separated from the patch.

5. (canceled)

6. (canceled)

7. The system of claim 1 further comprising a sleep adjustment device in communication with the control unit and configured to generate the stimulus based on the second signal.

8. The system of claim 7 wherein the sleep adjustment device is part of an adjustable bed.

9. The system of claim 8 wherein the stimulus generated by the sleep adjustment device results in a change in an angle of a mattress of the adjustable bed.

10. The system of claim 7 wherein the sleep adjustment device is part of a mattress.

11. (canceled)

12. The system of claim 10 wherein the sleep adjustment device is implemented as an integral part of the mattress, or as a pad configured to be placed on the mattress.

13. The system of claim 7 wherein the stimulus generated by the sleep adjustment device is a mechanical stimulus.

14. The system of claim 13 wherein the sleep adjustment device includes a plurality of actuator elements implemented such that one or more selected ones of the actuator elements are activated based on the second signal.

15. The system of claim 14 wherein each of the actuator elements is configured to provide a massaging vibration or a poking action as the stimulus when selected.

16. The system of claim 14 wherein the selected one or more ones of the actuator elements includes all of the actuator elements.

17. The system of claim 14 wherein the sleep adjustment device is further configured to sense sleeping position of the user, and the one or more actuator elements are selected based at least in part on the sensed sleeping position of the user.

18. The system of claim 17 wherein the one or more actuator elements are selected to induce a change in sleeping position of the user from the sensed sleeping position to a new sleeping position having a lower likelihood of snoring.

19. The system of claim 18 wherein the new position having the lower likelihood of snoring is determined based on a profile data associated with the user, the profile data included in or accessible by the control unit.

20. The system of claim 19 wherein the control unit includes a processor configured to build the profile data and/or to utilize the profile data.

21. The system of claim 20 wherein the processor is further configured to execute an algorithm including an artificial intelligence algorithm.

22. The system of claim 17 wherein the sleep adjustment device includes a plurality of pressure sensors.

23. The system of claim 22 wherein the plurality of pressure sensors are co-located with respective ones of the plurality of actuator elements.

24. A system for reducing snoring activity, the system comprising:
non-transitory computer readable medium including data representative of a sleep profile of a person, the sleep profile obtained from use of one or more patches each configured to be secured to a skin of the person and sense snoring of the person, such that the sleep profile includes information about a snoring event during which the person is likely to snore;
a control unit in communication with the non-transitory computer readable medium and configured to generate a sleep plan for the person based at least in part on the information about the snoring event, the sleep plan including a control signal; and
a sleep adjustment device configured to receive the control signal and provide a stimulus to the person, the stimulus selected to reduce the likelihood of snoring during a period associated with the snoring event.

25. (canceled)

26. (canceled)

27. (canceled)

28. (canceled)

29. (canceled)

30. (canceled)

31. (canceled)

32. (canceled)

33. (canceled)

34. (canceled)

35. A patch comprising:
a wearable component configured to allow the patch to be worn on a user; and
a sensor component implemented relative to the wearable component, the sensor component including a transducer configured to generate an electrical signal based on a snoring sound.

36-61. (canceled)
